(19) United States
(12) Patent Application Publication
(10) Pub. No.: US 2024/0376476 A1
(43) Pub. Date: Nov. 14, 2024

(54) NUCLEIC ACID TARGETING PROPROTEIN CONVERTASE SUBTILISIN/KEXIN 9 AND USES THEREOF

(71) Applicant: SynerK Biotech Limited, Hong Kong (HK)

(72) Inventors: Weiwen Jiang, Winchester, MA (US); Dong Yu, Westboro, MA (US); Jimmy Xibai Tang, Hong Kong (HK)

(21) Appl. No.: 18/196,080

(22) Filed: May 11, 2023

(51) Int. Cl. C12N 15/113; A61K 47/54

(52) U.S. Cl. CPC C12N 15/1137; A61K 47/549; C12Y 304/21061; C12N 2310/14

(57) ABSTRACT

The present disclosure relates to the technical field of biotechnology, and discloses a nucleic acid targeting proprotein convertase subtilisin/kexin 9 and uses thereof. The nucleic acid provided by the present disclosure can effectively inhibit the expression of PCSK9, thereby being capable of treating and/or preventing hyperlipidemia related diseases.

### SEQUENCE LISTING

The sequence listing submitted via EFS, in compliance with 37 CFR § 1.52 (e) (5), is incorporated herein by reference. The sequence listing XML file submitted via EFS contains the file “42693003USSeqList.xml”, created on Sep. 19, 2023, which is 169,204 bytes in size.

### FIELD

The present disclosure relates to the technical field of biotechnology, in particular to a nucleic acid targeting proprotein convertase subtilisin/kexin 9 and uses thereof.

### BACKGROUND

The proprotein convertase subtilisin/kexin 9 (PCSK9, also known as FH3, FHCL3, HCHOLA3, LDLCQ1, NARC-1, NARCI, PC9) is the 9th member of the proprotein convertase subtilysin family. The human PCSK9 gene, located at chromosome 1p3213, is approximately 22 kb in length and contains 12 exons, encodes a pro-keratin convertase including 692 amino acid residues, and is formed by connecting a signal peptide (SP1-30), a pre-structural domain (31-152), a catalytic domain (153-452) and a carboxy-terminal domain (526-692) sequentially; it is mainly expressed in liver, kidney and small intestine (Stoekenbroek R et al., Nat Rev Endocrinol. 15:52-62, 2019).

After secretion of the PCSK9 into the extracellular, the PCSK9 binds with the low-density lipoprotein (LDL) receptor on the cell surface, the PCSK9-LDL receptor complex enters into lysosome for degradation, resulting in the decreased LDL receptor on the cell surface, i.e., the PCSK9 level is inversely correlated with the LDL receptor (Park S et al. J Biol chem. 279:50630-50638, 2004; Shapiro M et al. Circulation Res. 122:1420-1438). Studies have shown that loss-of-mutation function of PCSK9 gene may lead to a significant decrease in LDL-C level and the incidence of coronary heart disease in different human species (Berge K et al. Arterioscler Thromb Vasc Biol. 26:1094-1100, 2006; Cohen J et al. Nat. Genet. 37:161-165, 2005; Horton J et al. Trends Biochem Sci. 32 (2): 71-77, 2009).

Hyperlipidemia is a major risk factor for the atherosclerotic cardiovascular disease, 60% of coronary atherosclerotic heart disease and 40% of ischemic stroke are caused by a prolonged elevation of low-density lipoprotein cholesterol (LDL-C) (Stone NJ et al. J Am Coll Cardiol. 63:2889-2934, 2014). Statins are still the main force in lowering LDL-C clinically at present, but the LDL-C of the 60-70% patients administered with statins still fails to reach the desirable level (Barkas F et al. Angiology. 66:346-353, 2015; Krahenhuhl S et al. Drugs. 76:1175-1190, 2016). In view of the significant effect of inhibiting PCSK9 in reducing LDL-C and the incidence of coronary heart disease, several therapeutic regimens are under development of drugs that block PCSK9 in order to reduce LDL-C and the incidence of coronary heart disease. Anti-PCSK9 monoclonal antibodies can effectively prevent PCSK9 from binding to LDL-R, allowing more LDL-R to be expressed by the liver, thereby lowering LDL-C level of plasma (Mullard A. Nat Rev Drug Discov. 11:817-819, 2012; Rosenson RS et al. J Am Coll Cardiol. 72:314-329, 2018). PCSK9 antibodies are currently clinically administered by 1-2 injections, typically 1 month (Zhang XL et al. BMC Med. 13:123-123, 2015), can effectively reduce the LDL-C.

Small interfering RNA (siRNA) molecules can reduce PCSK9 level (Fizgerald K et al. Lancet. 383:60-68, 2014). The siRNA molecules utilize the intracellular naturally occurring RNA interference (RNAi) pathway, bind to the intracellular RNA Induced Silencing Complex (RISC) to specifically cleave the messenger RNA (mRNA) molecules encoding PCSK 9. The cleaved mRNA will be degraded and cannot be translated into protein, which in turn results in the reduced protein level of PCSK 9. Therefore, it has important significance to study effective siRNA molecules to inhibit PCSK9 and thereby performing treatment for the related diseases.

### SUMMARY

The present disclosure aims to overcome the problems in the prior art, and provides a novel nucleic acid targeted to PCSK9 and uses thereof.

A first aspect of the present disclosure provides a nucleic acid comprising a sense strand and an antisense strand, wherein the sense strand contains a sequence having at least 80% sequence identity with a sequence set forth in SEQ ID NO:1, SEQ ID NO:3, SEQ ID NO:5, SEQ ID NO: 7, SEQ ID NO:9, SEQ ID NO: 11, SEQ ID NO: 13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55 or SEQ ID NO:57; the antisense strand contains a sequence having at least 80% sequence identity with a sequence set forth in SEQ ID NO:2, SEQ ID NO:4, SEQ ID NO:6, SEQ ID NO:8, SEQ ID NO: 10, SEQ ID NO:12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:32, SEQ ID NO: 34, SEQ ID NO:36, SEQ ID NO:38, SEQ ID NO:40, SEQ ID NO:42, SEQ ID NO:44, SEQ ID NO: 46, SEQ ID NO:48, SEQ ID NO:50, SEQ ID NO:52, SEQ ID NO:54, SEQ ID NO:56 or SEQ ID NO: 58.

A second aspect of the present disclosure provides a targeted drug delivery system comprising a target group, a linkage group, and the aforementioned nucleic acid connected with the target group via the linkage group.

A third aspect of the present disclosure provides a pharmaceutical composition comprising the aforementioned nucleic acid or targeted drug delivery system and a pharmaceutically acceptable carrier.

A fourth aspect of the present disclosure provides an use of the aforementioned nucleic acid, targeted drug delivery system or pharmaceutical composition in manufacture a medicament for treating and/or preventing hyperlipidemia related diseases.

A fifth aspect of the present disclosure provides an use of the aforementioned nucleic acid,

targeted drug delivery system or pharmaceutical composition in manufacture a medicament for reducing the expression level of proprotein convertase subtilisin/kexin 9.

The nucleic acids of the present disclosure are capable of effectively reducing the PCSK9 levels, have a stronger safety and longer half-life than antibodies, and are not prone to cause drug immunogenicity.

### BRIEF DESCRIPTION OF THE DRAWINGS

FIG. 1 illustrates an effect of siRNA in reducing serum PCSK9 protein in humanized PCSK9 mice;

FIG. 2 illustrates an effect of siRNA in reducing liver PCSK9 mRNA expression in humanized PCSK9 mice;

FIG. 3 illustrates an effect of siRNA dose-dependently decreasing serum PCSK9 concentration of humanized PCSK9 mice.

### DETAILED DESCRIPTION

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point value of the various ranges, and the individual point values may be combined with one another to produce one or more new numerical ranges, which should be deemed have been specifically disclosed herein.

The present disclosure provides a (modified or unmodified) nucleic acid comprising a sense strand and an antisense strand, the sense strand contains a sequence having at least 80% sequence identity with a sequence set forth in SEQ ID NO:1, SEQ ID NO:3, SEQ ID NO:5, SEQ ID NO: 7, SEQ ID NO:9, SEQ ID NO: 11, SEQ ID NO:13, SEQ ID NO: 15, SEQ ID NO: 17, SEQ ID NO: 19, SEQ ID NO:21, SEQ ID NO:23, SEQ ID NO:25, SEQ ID NO:27, SEQ ID NO:29, SEQ

ID NO: 31, SEQ ID NO:33, SEQ ID NO:35, SEQ ID NO:37, SEQ ID NO:39, SEQ ID NO:41, SEQ ID NO: 43, SEQ ID NO:45, SEQ ID NO:47, SEQ ID NO:49, SEQ ID NO:51, SEQ ID NO:53, SEQ ID NO: 55 or SEQ ID NO:57; the antisense strand contains a sequence having at least 80% sequence identity with a sequence set forth in SEQ ID NO:2, SEQ ID NO:4, SEQ ID NO:6, SEQ ID NO:8,SEQ ID NO:10, SEQ ID NO: 12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO: 58.

Wherein a sequence having at least 80% sequence identity refers to the circumstance of including 0, 1, 2, 3 or 4 different bases with the referred sequence, as well as the circumstance of additionally binding with more bases on the basis of 0, 1, 2, 3 or 4 different bases with the referred sequence. Furthermore, the different bases may be located anywhere in the referred sequence, but preferably, along the 5′-3′ direction, the different bases in the sense strand are the last 1-4 bases, and the different bases in the antisense strand are the first 1-4 bases.

More preferably, the sense strand has 16-30 (e.g., 16, 17, 18, 19, 20, 21, 22, 23, 24, 25, 26, 27, 28, 29, 30) nucleotides (bases). More preferably, the antisense strand has 16-30 (e.g., 16, 17, 18, 19, 20, 21, 22, 23, 24, 25, 26, 27, 28, 29, 30) nucleotides (bases).

In the present disclosure, the sense strand and the antisense strand may have the same or different length.

All the nucleotide acid groups in the aforesaid nucleic acid may be chemically unmodified, or contain at least one modified nucleotide group, and the modification may be on a random site of the nucleotide.

In preferred embodiments of the present disclosure, as shown in Table 1, the nucleic acid is at least one selected from siRNA-1 having a sense strand sequence of SEQ ID NO:1 and an antisense strand sequence of SEQ ID NO:2, siRNA-2 having a sense strand sequence of SEQ ID NO: 3 and an antisense strand sequence of SEQ ID NO:4, siRNA-3 having a sense strand sequence of SEQ ID NO:5 and an antisense strand sequence of SEQ ID NO:6, siRNA-4 having a sense strand sequence of SEQ ID NO:7 and an antisense strand sequence of SEQ ID NO:8, siRNA-5 having a sense strand sequence of SEQ ID NO:9 and an antisense strand sequence of SEQ ID NO:10, siRNA-6 having a sense strand sequence of SEQ ID NO:11 and an antisense strand sequence of SEQ ID NO: 12, siRNA-7 having a sense strand sequence of SEQ ID NO: 13 and an antisense strand sequence of SEQ ID NO:14, siRNA-8 having a sense strand sequence of SEQ ID NO:15 and an antisense strand sequence of SEQ ID NO:16, siRNA-9 having a sense strand sequence of SEQ ID NO: 17 and an antisense strand sequence of SEQ ID NO:18, siRNA-10 having a sense strand sequence of SEQ ID NO: 19 and an antisense strand sequence of SEQ ID NO:20, siRNA-11 having a sense strand sequence of SEQ ID NO:21 and an antisense strand sequence of SEQ ID NO:22, siRNA-12 having a sense strand sequence of SEQ ID NO:23 and an antisense strand sequence of SEQ ID NO:24, siRNA-13 having a sense strand sequence of SEQ ID NO:25 and an antisense strand sequence of SEQ ID NO:26, siRNA-14 having a sense strand sequence of SEQ ID NO:27 and an antisense strand sequence of SEQ ID NO:28, siRNA-15 having a sense strand sequence of

SEQ ID NO:29 and an antisense strand sequence of SEQ ID NO:30, siRNA-16 having a sense strand sequence of SEQ ID NO:31 and an antisense strand sequence of SEQ ID NO:32, siRNA-17 having a sense strand sequence of SEQ ID NO:33 and an antisense strand sequence of SEQ ID NO: 34, siRNA-18 having a sense strand sequence of SEQ ID NO:35 and an antisense strand sequence of SEQ ID NO:36, siRNA-19 having a sense strand sequence of SEQ ID NO:37 and an antisense strand sequence of SEQ ID NO:38, siRNA-20 having a sense strand sequence of SEQ ID NO: 39 and an antisense strand sequence of SEQ ID NO:40, siRNA-21 having a sense strand sequence of SEQ ID NO:41 and an antisense strand sequence of SEQ ID NO:42, siRNA-22 having a sense strand sequence of SEQ ID NO:43 and an antisense strand sequence of SEQ ID NO:44, siRNA-23 having a sense strand sequence of SEQ ID NO:45 and an antisense strand sequence of SEQ ID NO:46, siRNA-24 having a sense strand sequence of SEQ ID NO:47 and an antisense strand sequence of SEQ ID NO:48, siRNA-25 having a sense strand sequence of SEQ ID NO:49 and an antisense strand sequence of SEQ ID NO:50, siRNA-26 having a sense strand sequence of SEQ ID NO:51 and an antisense strand sequence of SEQ ID NO:52, siRNA-27 having a sense strand sequence of SEQ ID NO:53 and an antisense strand sequence of SEQ ID NO:54, siRNA-28 having a sense strand sequence of SEQ ID NO:55 and an antisense strand sequence of SEQ ID NO: 56, siRNA-29 having a sense strand sequence of SEQ ID NO:57 and an antisense strand sequence of SEQ ID NO: 58.

The nucleic acid according to the present disclosure, wherein the nucleic acid comprises a nucleotide group as the basic structural unit, the nucleotide group comprising a phosphate group, a ribose group and a base group, preferably the nucleic acid comprises at least one modified nucleotide group. The modification will not result in the functional loss of the nucleic acid inhibiting the PCSK9, or the modified nucleic acid has an efficiency of inhibiting PCSK9 not less than 50% (e.g., 50%, 51%, 52%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%) of the nucleic acid before modification.

The nucleic acid according to the present disclosure, wherein the modified nucleotide group is a nucleotide group in which the phosphate group and/or the ribose group is modified. The site with a modification may be at least the site 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 of the nucleotides at the site 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 of the sense strand and/or the antisense strand.

For example, modification of the phosphate group refers to the modification of oxygen in the phosphate group, including phosphorothioate modification and boranophosphate modification, etc. The oxygen in the phosphate group is substituted by sulfur, borane, amidogen, alkyl or alkoxy, respectively, as shown in the formulae below. Each of the modifications can stabilize structure of the nucleic acid, maintain high specificity and high affinity for the base pairing.

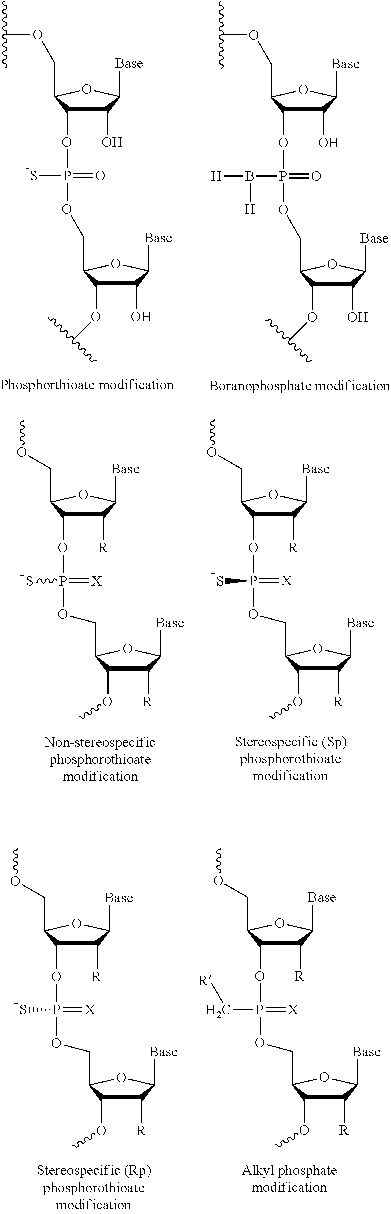

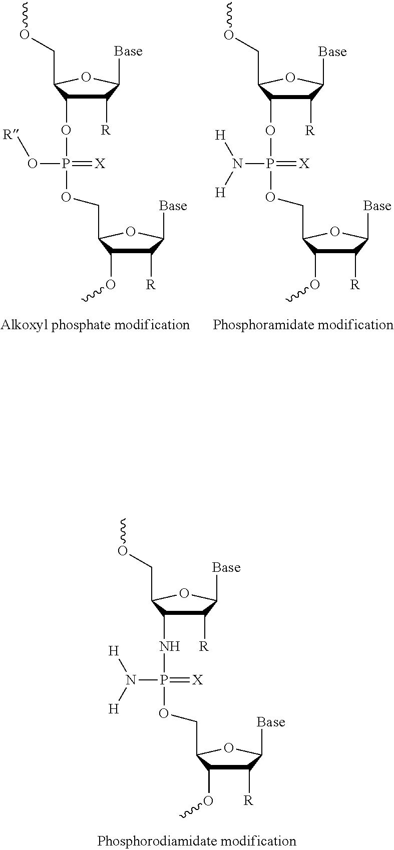

In the above structural formula, Base denotes the base A, U, C, G or T. X may be oxygen (O) or sulfur(S). R may be the same or different in the above structure, such as hydrogen (H), fluorine (F), methoxyl (OME) or Methoxyethyl (MOE), hydroxyl, allyl, ethylamino, propargyl, amino, cyanoethyl, acetyl etc.; R′and R″ may each independently be hydrogen (H), methyl (CH₃), ethyl (CH₂CH₃), propyl (CH₂CH₂CH₃), isopropyl (CH(CH₃)₂), allyl, propargyl, acyloxybenzyl and acyloxyethyl.

Modification of the ribose group refers to modification of the 2′-hydroxyl group (2′-OH) in the ribose group. After introducing certain substituents (e.g., methoxyl or fluorine) at the 2′-hydroxyl of the ribose group, the nucleic acid is not readily cleaved by ribonuclease, thereby enhancing stability of the nucleic acid and allowing the nucleic acid to be more resistant to hydrolysis by the nuclease. Modifications of the 2′-hydroxyl group in the nucleotide pentose include 2′-fluoro modification (e.g., 2′-arabino-fluoro modification), 2′-methoxy modification (2′- OME), 2′-methoxyethyl modification (2′-MOE), 2′-2,4-dinitrophenol modification (2-DNP modification), 2′,4′-constrained ethyl modification, 2′-Amino modification, 2′-Deoxy modification, BNA, acyclic nucleic acid modification, mal-positioned nucleic acid modification, L-type nucleic acid modification and the like. BNA (internal ring bridged nucleotide) refers to a constrained or inaccessible nucleotide. BNA can contain a bridging structure of a five-, six-, or seven-membered ring. The bridge is typically incorporated into the 2′-, 4′-position of the ribose ring to provide the 2′,4′-BNA nucleotides such as locked ethyl modification (LNA), constrained ethyl modification (ENA), and constrained ethyl bicyclic nucleic acid modification (cET BNA). Acyclic nucleic acid is a nucleotide formed after the ribose ring of the nucleotide is opened, such as unlocked nucleic acid (UNA) nucleotides and glycerol nucleic acid (GNA) nucleotides. Mal-positioned nucleic acid modification refers to a 3′,5′-phosphate bond linkage substituted by the 2′,5′-phosphate bond linkage. L-type nucleic acid modification refers to a naturally occurring D-type nucleic acid being substituted by its mirror-stereoscopic counterpart L-type nucleic acid.

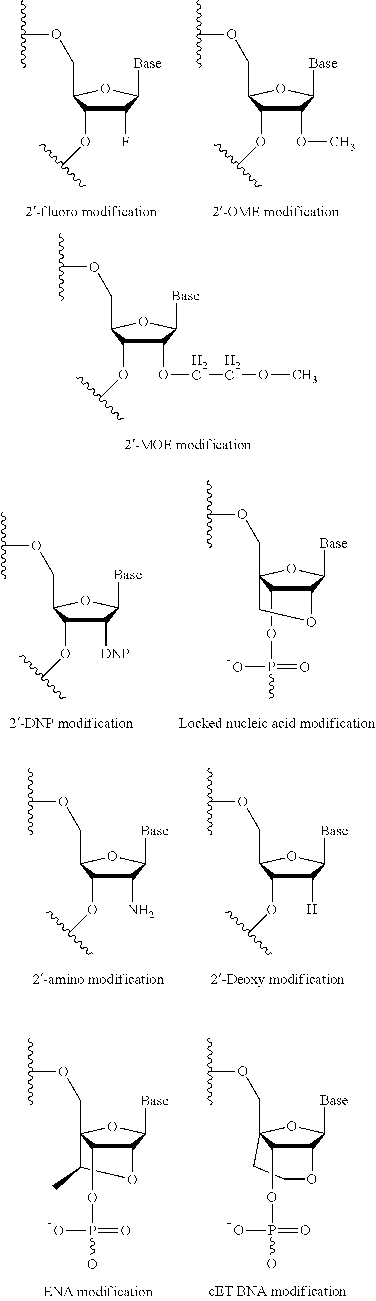

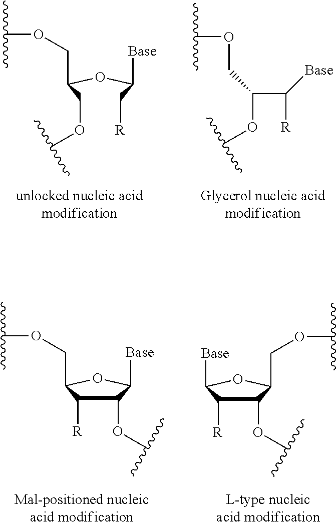

In the above structural formula, Base denotes the base A, U, C, G or T. R may be the same or different in the above structure, such as hydrogen (H), fluorine (F), methoxyl (OME) or Methoxyethyl (MOE), hydroxyl, allyl, ethylamino, propargyl, cyanoethyl and acetyl.

The nucleic acid according to the present disclosure, wherein the nucleotide group in which the ribose group is modified is preferably the nucleotide group in which the 2′-OH of the ribose group is substituted by methoxyl or fluorine.

According to a particularly preferred embodiment of the present disclosure, wherein the sense strand of nucleic acid comprising the nucleotide group of uracil base or cytosine base, which is the nucleotide group in which the ribose group is modified, that is, the 2′-OH of the ribose group in the sense strand of nucleic acid comprising the nucleotide group of uracil base or cytosine base is substituted by methoxyl or fluorine. More preferably, the 3′-end of both the sense strand and antisense strand of nucleic acid may be linked with dTdT; alternatively, the 3′-end of the antisense strand of nucleic acid may be linked with AA or UU, or any combination of two nucleic acids (the nucleic acids may be but are not limited to CC, GG or UG), to provide the sequence with specificity as inducement to mRNA degradation. Nucleic acids with such modifications exhibit more excellent in vivo inhibitory effect, and said modifications may further reduce the in vivo immunogenicity of the nucleic acids of the present disclosure.

The nucleic acid of the present disclosure may further comprise the modification in which a monophosphate nucleoside is linked to the 5′-end of the antisense strand. The 5′-monophosphate at the end of the guide strand of the siRNA is important for RISC recognition. Wherein phosphorylation of the 5′-hydroxyl group plays a certain role on whether the siRNA can be effectively loaded on the intracellular Ago2. The monophosphate at 5′-end of the guide strand in the siRNA has interaction with Argonaute-2 (Ago2) through the Hydrogen bond, in order to ensure accurate targeting and precise cleavage of the mRNA target. Several derivatives of the 5′-monophosphate nucleosides are commonly used, this type of derivative of the phosphate nucleoside has been proven to exhibit certain stability in the biological metabolism medium, and to play a certain role in facilitating the loading of siRNA guide strand on the intracellular Ago2 (Nucleic Acids Research, 2015, 43, 2993-3011). The nucleic acid according to the present disclosure, wherein the trans-vinyl phosphate (VP) is preferably the first choice, the nucleic acid may comprise derivatives of the monophosphate nucleoside other than those mentioned above.

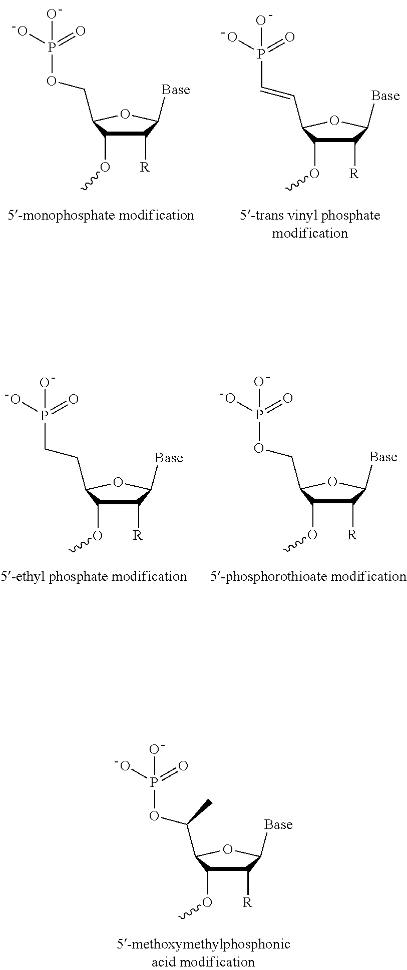

In the above structural formula, Base denotes the base A, U, C, G or T. R may be the same or different in the above structure, such as hydrogen (H), fluorine (F), methoxyl (OME) or methoxyethyl (MOE), hydroxyl, allyl, ethylamino, propargyl, cyanoethyl, amino and acetyl.

According to the preferred embodiments of the present disclosure, the base sequences of the modified nucleic acids and the modification modes are as shown in Table 2 (f, s, underlined), i.e., the sense strand and antisense strand of the modified nucleic acids siRNA-9, siRNA-13, siRNA-16, siRNA-19 and siRNA-23 have the modifications indicated by f, s and underline as shown in Table 2, and the 3′ end of the antisense strand is further connected with UU.

In a particularly preferred embodiment, the nucleic acid is selected from siRNA-23 having a sense strand sequence of SEQ ID NO: 45 and an antisense strand sequence of SEQ ID NO: 46, and having the modifications at the nucleotide as follows:

5′-GsCsCAAGCfCUfCUfUCUUACUUCA-3′
5′-UsGfsAAfGUfAAGAAGAGfGCfUUGGCsUsU-3′

wherein the lowercase letter f indicates that the nucleotide adjacent to the left side of the letter f is a 2′-fluoro modified nucleotide (i.e., the 2′-OH of the pentose in the nucleotide is substituted by fluorine); the lowercase letter s indicates that two nucleotides adjacent to the left side and right side of the letter s are connected through a thiophosphate diester bond (i.e., the non- bridging oxygen atom in the phosphodiester bond is substituted by sulfur atom); the underlined nucleotide indicates that the 2′ hydroxyl group of the nucleotide is substituted by methoxyl group. Such modifications result in higher in vivo activity of the nucleic acid (siRNA).

The nucleic acid according to the present disclosure can be obtained by conventional methods in the art, for example through the solid-phase synthesis and solution-phase synthesis, wherein the solid-phase synthesis has the commercial customization service, thus it is commercially available. The modified nucleotide group can be introduced by means of the nucleotide monomer having the corresponding modification.

Based on the synthesized nucleic acid (siRNA) mentioned above, the present disclosure can further construct an expression plasmid of the shRNA having the identical or similar function with the siRNA, and the method for constructing the expression plasmid is well-known among those skilled in the art, the content will not be described in detail herein.

The present disclosure further provides a target gene sequence of a nucleic acid as described above. In some embodiments, the target gene sequence is set forth in any item of column 2 of Table 1.

TABLE 1

| | | Sense | Antisense |
|---|---|---|---|
| | Target | strand | strand |
| | gene | sequence | sequence |
| No. | sites | (5′-3′) | (5′-3′) |
| 1 | 3317-3337 | AGCUCGGUGAGUG | UCUGCCAUCACUCAC |
| | | AUGGCAGA (1) | CGAGCU (2) |
| 2 | 3318-3336 | GCUCGGUGAGUGA | CUGCCAUCACUCACC |
| | | UGGCAG (3) | GAGC (4) |
| 3 | 3183-3201 | GCUCACACAGCAG | CAGUUCCUGCUGUGU |
| | | GAACUG (5) | GAGC (6) |
| 4 | 2117-2135 | AGUCAAGGAGCAU | GAUUCCAUGCUCCUU |
| | | GGAAUC (7) | GACU (8) |
| 5 | 2730-2748 | UCCUCAGGUCUCC | CUUGGUGGAGACCUG |
| | | ACCAAG (9) | AGGA (10) |
| 6 | 2890-2908 | UGGAGGCUUAGCU | CCAGAAAGCUAAGCC |
| | | UUCUGG (11) | UCCA (12) |
| 7 | 3199-3217 | CUGAGCCAGAAAC | AUCUGCGUUUCUGGC |
| | | GCAGAU (13) | UCAG (14) |
| 8 | 1889-1907 | CUGCAGCGUCCAC | AGCUGUGUGGACGCU |
| | | ACAGCU (15) | GCAG (16) |
| 9 | 3318-3338 | GCUCGGUGAGUGA | UUCUGCCAUCACUCA |
| | | UGGCAGAA (17) | CCGAGC (18) |
| 10 | 626-644 | GCAUGUCUUCCAU | AAGGCCAUGGAAGAC |
| | | GGCCUU (19) | AUGC (20) |
| 11 | 827-845 | GGUGGAGGUGUAU | UAGGAGAUACACCUC |
| | | CUCCUA (21) | CACC (22) |
| 12 | 893-911 | GGUCACCGACUUC | AUUCUCGAAGUCGGU |
| | | GAGAAU (23) | GACC (24) |
| 13 | 893-913 | GGUCACCGACUUC | ACAUUCUCGAAGUCG |
| | | GAGAAUGU (25) | GUGACC (26) |
| 14 | 3039-3059 | AGCCAUCACCUAG | AGUCAGUCCUAGGUG |
| | | GACUGACU (27) | AUGGCU (28) |
| 15 | 2612-2632 | GCAUUUCACCAUU | CCUGUUUGAAUGGUG |
| | | CAAACAGG (29) | AAAUGC (30) |
| 16 | 3466-3484 | CCCAAGCAAGCAG | AAAUGUCUGCUUGCU |
| | | ACAUUU (31) | UGGG (32) |
| 17 | 3433-3451 | CCAACAACUGUCC | AAGGAGGGACAGUUG |
| | | CUCCUU (33) | UUGG (34) |
| 18 | 3399-3418 | GGAGAUGCUUCUA | AUGCCUUAGAAGCAU |
| | | AGGCAU (35) | CUCC (36) |
| 19 | 3192-3210 | GCAGGAACUGAGC | UUUCUGGCUCAGUUC |
| | | CAGAAA (37) | CUGC (38) |
| 20 | 2900-2918 | GCUUUCUGGAUGG | UAGAUGCCAUCCAGA |
| | | CAUCUA (39) | AAGC (40) |
| 21 | 2880-2898 | CCCUGAUUAAUGG | AAGCCUCCAUUAAUC |
| | | AGGCUU (41) | AGGG (42) |
| 22 | 2744-2762 | CCAAGGAGGCAGG | AAGAAUCCUGCCUCC |
| | | AUUCUU (43) | UUGG (44) |
| 23 | 3233-3253 | GCCAAGCCUCUUC | UGAAGUAAGAAGAGG |
| | | UUACUUCA (45) | CUUGGC (46) |
| 24 | 2969-2989 | GGGCAUUUCACCA | AGUAAAGGUGGCUCA |
| | | UUCAAACA (47) | GGUUUC (48) |
| 25 | 2978-2998 | GCCACCUUUACUC | AUAGAGCAGAGUAAA |
| | | UGCUCUAU (49) | GGUGGC (50) |
| 26 | 3471-3489 | GCAAGCAGACAUU | AAGAUAAAUGUCUGC |
| | | UAUCUU (51) | UUGC (52) |
| 27 | 2610-2628 | GGGCAUUUCACCA | UUUGAAUGGUGAAAU |
| | | UUCAAA (53) | GCCC (54) |
| 28 | 2612-2630 | GCAUUUCACCAUU | UGUUUGAAUGGUGAA |
| | | CAAACA (55) | AUGC (56) |
| 29 | 2722-2740 | GCAUUCAAUCCUC | AGACCUGAGGAUUGA |
| | | AGGUCU (57) | AUGC (58) |

Note:
“(1)” recited in columns 3-4 denotes SEQ ID NO: 1, the number “3317-3337” recited in column 2 represents nucleotides at sites 3317-3337 in PCSK9 gene sequence, and the like.

The coding sequence of human PCSK9 (NM_174936.4, SEQ ID NO: 59):

| | |
|---|---|
| 1 | agcgacgtcg aggcgctcat ggttgcaggc gggcgccgcc gttcagttca gggtctgagc |
| 61 | ctggaggagt gagccaggca gtgagactgg ctcgggcggg ccgggacgcg tcgttgcagc |
| 121 | agcggctccc agctcccagc caggattccg cgcgcccctt cacgcgccct gctcctgaac |
| 181 | ttcagctcct gcacagtcct ccccaccgca aggctcaagg cgccgccggc gtggaccgcg |
| 241 | cacggcctct aggtctcctc gccaggacag caacctctcc cctggccctc atgggcaccg |
| 301 | tcagctccag gcggtcctgg tggccgctgc cactgctgct gctgctgctg ctgctcctgg |
| 361 | gtcccgcggg cgcccgtgcg caggaggacg aggacggcga ctacgaggag ctggtgctag |
| 421 | ccttgcgttc cgaggaggac ggcctggccg aagcacccga gcacggaacc acagccacct |
| 481 | tccaccgctg cgccaaggat ccgtggaggt tgcctggcac ctacgtggtg gtgctgaagg |
| 541 | aggagaccca cctctcgcag tcagagcgca ctgcccgccg cctgcaggcc caggctgccc |
| 601 | gccggggata cctcaccaag atcctgcatg tcttccatgg ccttcttcct ggcttcctgg |
| 661 | tgaagatgag tggcgacctg ctggagctgg ccttgaagtt gccccatgtc gactacatcg |
| 721 | aggaggactc ctctgtcttt gcccagagca tcccgtggaa cctggagcgg attacccctc |
| 781 | cacggtaccg ggcggatgaa taccagcccc ccgacggagg cagcctggtg gaggtgtatc |
| 841 | tcctagacac cagcatacag agtgaccacc gggaaatcga gggcagggtc atggtcaccg |
| 901 | acttcgagaa tgtgcccgag gaggacggga cccgcttcca cagacaggcc agcaagtgtg |
| 961 | acagtcatgg cacccacctg gcaggggtgg tcagcggccg ggatgccggc gtggccaagg |
| 1021 | gtgccagcat gcgcagcctg cgcgtgctca actgccaagg gaagggcacg gttagcggca |
| 1081 | ccctcatagg cctggagttt attcggaaaa gccagctggt ccagcctgtg gggccactgg |
| 1141 | tggtgctgct gcccctggcg ggtgggtaca gccgcgtcct caacgccgcc tgccagcgcc |
| 1201 | tggcgagggc tggggtcgtg ctggtcaccg ctgccggcaa cttccgggac gatgcctgcc |
| 1261 | tctactcccc agcctcagct cccgaggtca tcacagttgg ggccaccaat gcccaagacc |
| 1321 | agccggtgac cctggggact ttggggacca actttggccg ctgtgtggac ctctttgccc |
| 1381 | caggggagga catcattggt gcctccagcg actgcagcac ctgctttgtg tcacagagtg |
| 1441 | ggacatcaca ggctgctgcc cacgtggctg gcattgcagc catgatgctg tctgccgagc |
| 1501 | cggagctcac cctggccgag ttgaggcaga gactgatcca cttctctgcc aaagatgtca |
| 1561 | tcaatgaggc ctggttccct gaggaccagc gggtactgac ccccaacctg gtggccgccc |
| 1621 | tgccccccag cacccatggg gcaggttggc agctgttttg caggactgta tggtcagcac |
| 1681 | actcggggcc tacacggatg gccacagccg tcgcccgctg cgccccagat gaggagctgc |
| 1741 | tgagctgctc cagtttctcc aggagtggga agcggcgggg cgagcgcatg gaggcccaag |
| 1801 | ggggcaagct ggtctgccgg gcccacaacg cttttggggg tgagggtgtc tacgccattg |
| 1861 | ccaggtgctg cctgctaccc caggccaact gcagcgtcca cacagctcca ccagctgagg |
| 1921 | ccagcatggg gacccgtgtc cactgccacc aacagggcca cgtcctcaca ggctgcagct |
| 1981 | cccactggga ggtggaggac cttggcaccc acaagccgcc tgtgctgagg ccacgaggtc |
| 2041 | agcccaacca gtgcgtgggc cacagggagg ccagcatcca cgcttcctgc tgccatgccc |
| 2101 | caggtctgga atgcaaagtc aaggagcatg gaatcccggc ccctcaggag caggtgaccg |
| 2161 | tggcctgcga ggagggctgg accctgactg gctgcagtgc cctccctggg acctcccacg |
| 2221 | tcctgggggc ctacgccgta gacaacacgt gtgtagtcag gagccgggac gtcagcacta |
| 2281 | caggcagcac cagcgaaggg gccgtgacag ccgttgccat ctgctgccgg agccggcacc |
| 2341 | tggcgcaggc ctcccaggag ctccagtgac agccccatcc caggatgggt gtctggggag |
| 2401 | ggtcaagggc tggggctgag ctttaaaatg gttccgactt gtccctctct cagccctcca |
| 2461 | tggcctggca cgaggggatg gggatgcttc cgcctttccg gggctgctgg cctggccctt |
| 2521 | gagtggggca gcctccttgc ctggaactca ctcactctgg gtgcctcctc cccaggtgga |
| 2581 | ggtgccagga agctccctcc ctcactgtgg ggcatttcac cattcaaaca ggtcgagctg |
| 2641 | tgctcgggtg ctgccagctg ctcccaatgt gccgatgtcc gtgggcagaa tgacttttat |
| 2701 | tgagctcttg ttccgtgcca ggcattcaat cctcaggtct ccaccaagga ggcaggattc |
| 2761 | ttcccatgga taggggaggg ggcggtaggg gctgcaggga caaacatcgt tggggggtga |
| 2821 | gtgtgaaagg tgctgatggc cctcatctcc agctaactgt ggagaagccc ctgggggctc |
| 2881 | cctgattaat ggaggcttag ctttctggat ggcatctagc cagaggctgg agacaggtgc |
| 2941 | gcccctggtg gtcacaggct gtgccttggt ttcctgagcc acctttactc tgctctatgc |
| 3001 | caggctgtgc tagcaacacc caaaggtggc ctgcggggag ccatcaccta ggactgactc |
| 3061 | ggcagtgtgc agtggtgcat gcactgtctc agccaacccg ctccactacc cggcagggta |
| 3121 | cacattcgca cccctacttc acagaggaag aaacctggaa ccagaggggg cgtgcctgcc |
| 3181 | aagctcacac agcaggaact gagccagaaa cgcagattgg gctggctctg aagccaagcc |
| 3241 | tcttcttact tcacccggct gggctcctca tttttacggg taacagtgag gctgggaagg |
| 3301 | ggaacacaga ccaggaagct cggtgagtga tggcagaacg atgcctgcag gcatggaact |
| 3361 | ttttccgtta tcacccaggc ctgattcact ggcctggcgg agatgcttct aaggcatggt |
| 3421 | cgggggagag ggccaacaac tgtccctcct tgagcaccag ccccacccaa gcaagcagac |
| 3481 | atttatcttt tgggtctgtc ctctctgttg cctttttaca gccaactttt ctagacctgt |
| 3541 | tttgcttttg taacttgaag atatttattc tgggttttgt agcattttta ttaatatggt |
| 3601 | gactttttaa aataaaaaca aacaaacgtt gtcctaa |

The present disclosure also provides a targeted drug delivery system comprising a target group, a linkage group, and the aforementioned nucleic acid connected with the targeted group via the linkage group. Wherein the targeted group is capable of further improving targeting performance of the small nucleic acid, it may be provided by a monosaccharide (e.g., glucose, mannose, allose, altrose, galactose, galactosamine, N-acetylgalactosamine, talose, fructose, idose) and/or a polypeptide (e.g., protein, monoclonal antibody, nanoparticle). The linkage group may be selected from-O-[CH₂CH₂O]ₙ-, -[CH₂O]ₘ13 CONH—[CH₂]ₙO—, —O-[CH₂CH₂O]ₘ—CONH—[CH₂]ₙO—, O—[CH₂]ₘ—CONH— [CH₂H₂O]ₙO—. Wherein m and n each may independently be an integer from 1 to 10.

According to a preferred embodiment of the present disclosure, the targeted drug delivery system has a structure shown below, wherein Nu represents a nucleic acid (siRNA) of the present disclosure, wherein the compound moiety can be coupled with the 5′ end or the 3′ end of the sense strand of siRNA via a phosphodiester bond, or can be coupled with the 5′ end or the 3′ end of the antisense strand of siRNA via a phosphodiester bond. Specifically, the compound moiety can be synthesized with a nucleoside monomer or a nucleoside attached to a solid phase carrier under coupling reaction conditions and in the presence of a coupling reagent, thereby allowing the compound moiety to be attached to the nucleic acid via coupling reaction. The targeted drug delivery system can improve the cell penetrating capability of the nucleic acid drug (Nu) by using the structural characteristics on the left side thereof, thereby enhance the stability of Nu in cells, and the preparation process is simple and highly practical.

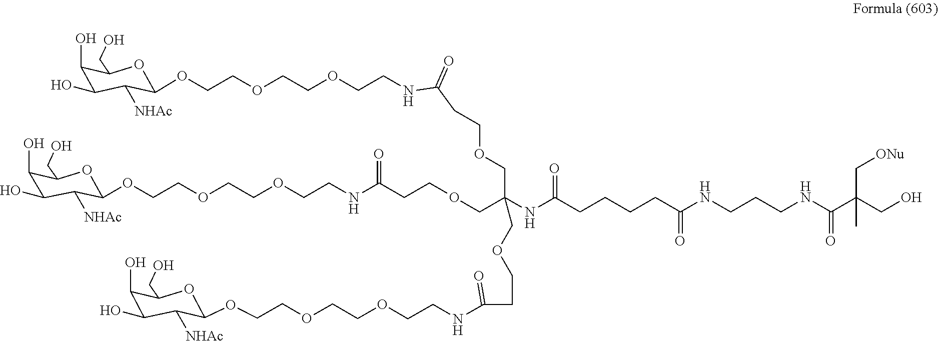

The present disclosure further provides a pharmaceutical composition comprising the aforementioned nucleic acid or targeted drug delivery system and a pharmaceutically acceptable carrier. The pharmaceutical composition can be prepared with the nucleic acid and the pharmaceutically acceptable carrier through conventional method. For example, the pharmaceutical composition may be an injection solution. The injection solution can be used for subcutaneous, intramuscular or intravenous injection.

The pharmaceutical composition according to the present disclosure, wherein the dosage

of nucleic acid or the targeted drug delivery system and the pharmaceutically acceptable carrier are not particularly defined, typically the amount of the pharmaceutically acceptable carrier may be within a range of 1-100,000 parts by weight (e.g. 1 part by weight, 5 parts by weight, 10 parts by weight, 50 parts by weight, 100 parts by weight, 500 parts by weight, 1,000 parts by weight, 5,000 parts by weight, 10,000 parts by weight, 50,000 parts by weight, 100,000 parts by weight or a random value between any two numerical values mentioned above) relative to 1 part by weight of the nucleic acid (or 1 part by weight of the targeted drug delivery system calculated in terms of the nucleic acid).

The pharmaceutical composition according to the present disclosure, wherein the pharmaceutically acceptable carrier may be various carrier conventionally used in the art, for example, the pharmaceutically acceptable carrier may include at least one of a pH buffer solution, a protective agent and an osmotic pressure conditioning agent. The pH buffer solution may be a tri (hydroxymethyl) aminomethane hydrochloride buffer having a pH of 7.5-8.5 and/or a phosphate buffer having a pH of 5.5-8.5, preferably a phosphate buffer having a pH of 5.5-8.5. The protective agent may be at least one of inositol, sorbitol and sucrose. The protective agent may be contained in an amount of 0.01-30 wt. % (e.g., 0.01 wt. %, 0.05 wt. %, 0.1 wt. %, 0.5 wt. %, 1 wt. %, 5 wt. %, 10 wt. %, 15 wt. %, 20 wt. %, 25 wt. %, 30 wt. % or a random value between any two numerical values thereof) based on the total weight of the pharmaceutical composition. The osmotic pressure conditioning agent may be sodium chloride and/or potassium chloride. The osmotic pressure conditioning agent is contained in an amount such that the osmotic pressure of the pharmaceutical composition is within a range of 200-700 milliosmols per kilogram. The amount of the osmotic pressure conditioning agent may be determined by those skilled in the art based on the desired osmotic pressure.

According to a preferred embodiment of the present disclosure, the pharmaceutically acceptable carrier is a liposome. The liposome may be any liposome capable of encapsulating nucleic acid, it may have a diameter of 25-1,000 nm, and may include but not limited to the cholesterol and analog or derivative thereof.

The dosage of the pharmaceutical composition according to the present disclosure may be

a conventional dosage in the art, the dosage may be determined according to various parameters, in particular the age, body weight and sex of a subject. For example, for female mice having a weight of 25-30 g at an age of 3-4 months, the pharmaceutical composition may be used in a dosage of 0.01-100 mg/kg body weight, preferably 1-10 mg/kg body weight, based on the amount of nucleic acid in the pharmaceutical composition.

The present disclosure also provides an use of the aforementioned nucleic acid, targeted drug delivery system or pharmaceutical composition in manufacture a medicament for treating and/or preventing hyperlipidemia related diseases. In the pharmaceutical composition for treating and/or preventing hyperlipidemia related diseases, the nucleic acid functions mainly through the mechanism of RNA interference.

The present disclosure further provides an use of the aforementioned nucleic acid, targeted drug delivery system or pharmaceutical composition in manufacture a medicament for reducing the expression level of proprotein convertase subtilisin/kexin 9 (e.g., in serum or liver).

The present disclosure also provides a method of inhibiting PCSK9, the method comprises administering the nucleic acid and/or pharmaceutical composition to a patient suffering from the hyperlipidemia related diseases. “inhibiting” refers to silencing the gene expression of PCSK 9. The patient may be a mammal, preferably a primate, more preferably a human. Administration may be performed through various routes, depending on whether the local or systemic treatment is required. The administration mode may be, but is not limited to, intravenous administration, intraarterial administration, subcutaneous administration, intraperitoneal administration, transdermal administration (e.g., by implantation of a device), and intra soft tissue administration. “Hyperlipidemia related diseases” may include coronary atherosclerosis heart disease,

ischemic stroke, arteriosclerosis cerebral infarction, hypertension, diabetes mellitus, nephropathy (or kidney disease), peripheral vascular diseases and the like.

In addition, the present disclosure further provides a method of inhibiting PCSK9 in vitro, the method comprises introducing the nucleic acid and/or pharmaceutical composition into a cell.

The present disclosure will describe in detail below with reference to examples. Unless

otherwise specified in the present disclosure, the reagents and culture media used therein are commercially available, the nucleic acid electrophoresis and other operations used in the present disclosure is performed in a conventional manner.

### Example 1

The siRNA-1 to siRNA-29 listed in Table 1 were obtained by a solid phase synthesis method. 0.5 ml of cell culture fluid (DMEM, 10% FBS) containing 10⁵ Hep3B cells was added into a 24-well cell culture dish, and cultured overnight in a cell culture vessel containing 5% CO₂ at the temperature of 37° C. Lipofectamine® RNAiMAX (1-2 microliters/well) and small interfering nucleic acid siRNA-1 to siRNA-29 as shown in Table 1 were added into the serum-free cell culture fluid, and the serum-free cell culture fluid was added into the cell culture wells such that the final concentration of small interfering nucleic acid per well was 33 nM or 100 nM, and continued to culture for 48 hours in the cell culture vessel containing 5% CO₂ at the temperature of 37° C. In order to extract RNA, the cell culture supernatant was pipetted completely, washed with PBS, and after pipetting completely, the RNA was extracted according to the operating instruction of the

RNAeasy Mini kit (QIAGEN, Article No. 74104). RT-PCR was performed according to the recommendation of High Capacity cDNA Reverse Transcription Kits (Thermo Fisher, Article No. 4368814), and 0.5 or 1 ug of RNA was contained in each reaction. The gene expression was quantified by using the real-time fluorescence PCR method, and the TaqMan probe of human PCSK9 was Hs00545399_m1, and the probe of the internal reference gene (human HPRT1) was Hs02800695_ml (Thermo Fisher Scientific, Waltham, MA, USA). The PCR conditions were 1 cycle for 20 sec at the temperature of 95° C., 40 cycles for 1 sec at 95° C. and 20 sec at 60° C., and the real-time fluorescence PCR instrument was StepOne Plus (Thermo Fisher). The PCSK9 gene expression was calculated based on the 2\hat{ }-ΔΔCt, and the human HPRT1 gene expression was used as the internal reference. The expression level of PCSK9 gene was expressed as a percentage of treated group vs. control group (using RNAiMAX only) (see Table 2).

TABLE 2

| No. | 33 nM (%) | 100 nM (%) |
|---|---|---|
| 1 | 38.9 | 32.0 |
| 2 | 43.5 | 59.1 |
| 3 | 28.1 | 25.7 |
| 4 | 22.4 | 21.6 |
| 5 | 55.4 | 56.2 |
| 6 | 37.6 | 62.9 |
| 7 | 39.8 | 59.8 |
| 8 | 85.1 | 111.7 |
| 9 | 34.1 | 31.2 |
| 10 | 27.7 | 30.5 |
| 11 | 73.4 | 70.7 |
| 12 | 61.7 | 58.7 |
| 13 | 15.7 | 11.5 |
| 14 | 18.6 | 17.4 |
| 15 | 16.6 | 13.7 |
| 16 | 16.0 | 12.8 |
| 17 | 54.1 | 47.8 |
| 18 | 20.7 | 17.1 |
| 19 | 22.1 | 16.9 |
| 20 | 18.0 | 15.8 |
| 21 | 16.4 | 12.7 |
| 22 | 23.6 | 12.6 |
| 23 | 22.8 | 18.4 |
| 24 | 63.5 | 52.2 |
| 25 | 24.4 | 16.7 |
| 26 | 23.4 | 16.2 |
| 27 | 20.6 | 20.3 |
| 28 | 28.2 | 17.0 |
| 29 | 33.5 | 18.6 |

### Example 2

(I) Preparation of siRNA drug according to the following steps.

### Synthesis of Conjugate (603A)

1. Compound 7 was prepared according to the following route:

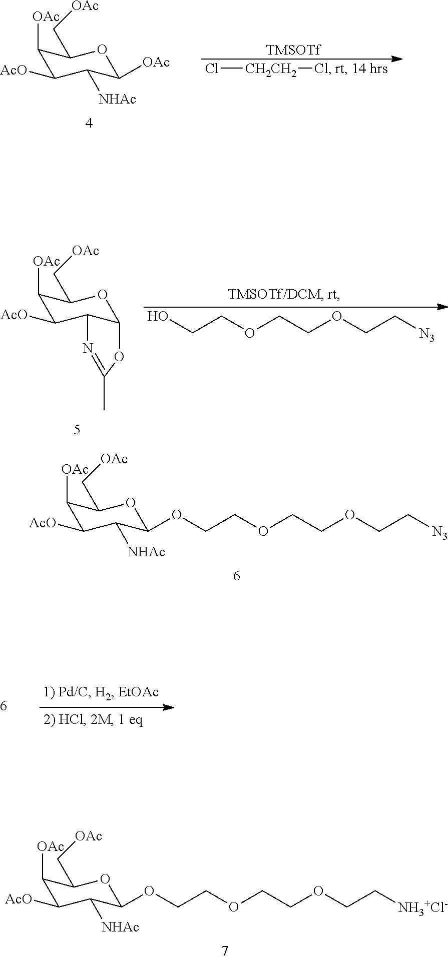

### Synthesis of Compound Oxazoline 5

N-acetylgalactosamine tetraacetate 4 (10 g, 25.68 mmol) was dissolved in dichloroethane (60 mL) at room temperature, trimethylsilyl trifluoromethanesulfonate (8.6 g, 38.66 mmol) was added to the aforesaid solution under the stirring condition, stirring was continued and the solution was heated to 50° C. After reaction at 50° C. for 2 hours, the heating was stopped, and the stirring was continued for 12 hours at room temperature. The solution was poured into ice water containing saturated sodium bicarbonate, extracted with dichloromethane, and the organic phase was subjected to washing with water. The organic phase was separated out, and dried after adding anhydrous sodium sulfate, and evaporated and dried in reduced pressure to obtain a brownish-yellow foamy syrup-shaped compound 5. The compound 5 was directly used in the next step.

### Synthesis of Compound 6

The compound oxazoline 5 (4.26 g, 12.9 mmol) was dissolved in dichloromethane (20 mL) at room temperature, and mixed with a solution of dry dichloromethane (20 mL) dissolved with 2-[2-(2-azidoethoxy) ethoxy] ethanol (3.4 g, 19 mmol) and stirred under the temperature condition of 0° C. Trimethylsilyl trifluoromethanesulfonate (TMSOTf, 1.4 g, 6.45 mmol) was added slowly at 0° C. into the solution and stirred for 1 hour. The mixed solution was stirred continuously for 14 hours at room temperature, the solution was then poured into ice water containing saturated sodium bicarbonate, extracted with dichloromethane (2×50 mL), and the organic phase was subjected to washing with water. The organic phase was separated out, and dried with anhydrous sodium sulfate, and rotary evaporated and concentrated in reduced pressure to a semi-dry state. Purification was further performed with a silica gel chromatographic column, a gradient elution was used, initially rinsed with a mixed solvent (containing ethyl acetate/methanol, 10:1, v/v), the product components were collected, and the solvent was drained under reduced pressure to obtain the near-white compound 6 (5.3g, 81%). ¹H NMR (CDCl₃): δ, 6.15 (d, 1H, NH), 5.32 (d, 1H, sugar-H-4′), 5.07 (dd, 1H, J=11.2 Hz, J=3.3 Hz, sugar-H-3′), 4.76 (d, 1H, J=8.6 Hz, sugar-H-1′), 4.17 (m, 3H, sugar-H-2′, sugar-H-6′), 3.91 (m, 2H, —CH₂O), 3.89 (m, 1H, suger-H-5′), 3.76-3.61 (m, 8H, —CH₂O), 3.47 (m, 2H, —CH2N3), 2.16 (s, 3H, —CH3, NHAc), 1.99, 2.00, 2.05 (3 xs, 9H, —CH_{3,} Ac). HRMS (ESI) m/z, C₂₀H₃₂N₄O₁₁ (M+H⁺). Theoretical value: 505.49, measured value: 505.20.

### Synthesis of Compound 7

Azide 6 (522 mg, 1.04 mmol) was dissolved in 10 mL of ethyl acetate, Pd/C (80 mg) was added into 30 mL of ethyl acetate under the protection of nitrogen gas. The reaction bottle was connected to a hydrogen balloon, subjected to multiple replacements with hydrogen gas, the reaction bottle was connected to a hydrogen balloon at room temperature, the reaction solution was continuously stirred for 3 hours. Pd/C was filtered by the Celite, and 0.5 mL of hydrochloric acid (2M) was added dropwise and slowly, and the solution was reacted under continuous stirring for 30 minutes under a reaction temperature of 0° C. 10 mL of acetonitrile was added into the reaction solution, and subjected to azeotropic decompression concentration for twice. The concentrated solution was mixed with dichloromethane (10 mL), and concentration under reduced pressure was further performed for twice, resulting in an oily foamed crude product 7 (500 mg), which was directly used in the next step without further purification. 1H NMR (CDC13): 8, 8.25 (m, 2H, —NH₂), 5.34 (d, 1H, sugar-H-4′), 5.21 (dd, 1H, J=11.2Hz, J=3.3Hz, sugar-H-3′), 4.91 (d, 1H, J=8.5 Hz, sugar-H-1′), 4.12 (m, 3H, sugar-H-6′, sugar-H-2′), 4.07 (m, 2H, sugar-H-5′, —NH), 3.76 (m, 2H, —CH20), 3.68 (m, 2H, —CH₂O), 3.61 (m, 2H,-CH2O), 3.58 (m, 4H, 2 x-CH2O), 3.20 (m, 2H, NH₂), 2.09 (s, 3H, —NHCO₂CH₃), 2.04, 1.96, 1.89 (3 x s, 9H, —CO₂CH₃). HRMS (ESI) m/z, C₂₀H₃₄N₂O₁₁ (M+H⁺). Theoretical values: 479.49, measured values: 479.20.

### 2. Compound 12 was prepared according to the following route:

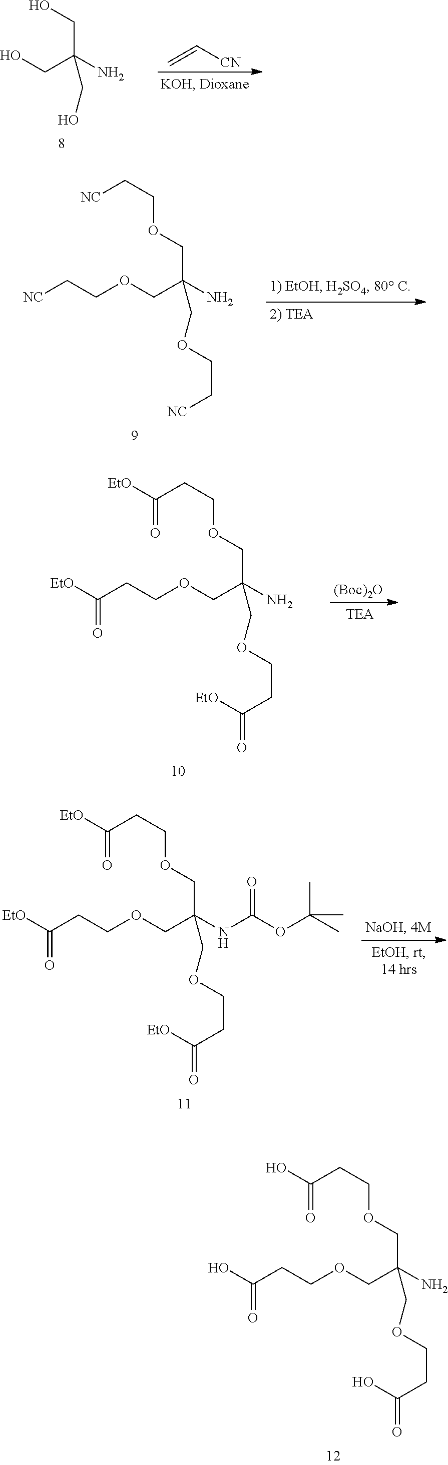

### Synthesis of Compound 9

Trihydroxymethyl aminomethane 8 (10 g, 82.6 mmol) was dissolved in 15 mL of dioxane, 1.26 mL of an aqueous potassium hydroxide solution with a concentration of 40 wt % was dropwise added into the reaction solution and stirred, 20 mL of dioxane was further added at room temperature. Acrylonitrile (18 mL, 272 mmol) was added dropwise and slowly to the reaction flask under the temperature of 0° C., and the entire dropwise adding process was maintained about 1 hour. The reaction solution was continuously stirred at room temperature for 24 hours. The reaction solution was poured into a saturated sodium chloride solution, and extracted with dichloromethane (2×50 mL), the organic phase was subjected to washing with water. The organic phase was separated out and dried with the added anhydrous sodium sulfate, and rotary evaporated and concentrated in reduced pressure to a semi-dry state. Purification was further performed with a silica gel chromatographic column, the purified product was first rinsed with dichloromethane and then rinsed with a solvent mixture (containing dichloromethane/methanol, 10:1, v/v), the product components were collected. The product components were subjected to rotary evaporation and concentration in reduced pressure to obtain a pale yellow oily substance 9 (20 g, 86%). ¹H NMR

(CDCl₃): δ, 3.68 (t, 6H, J=7.1 Hz, 3 x —CH₂O), 3.44 (s, 6H, 3 x —CH₂CNH₂), 2.61 (t, 6H, J=6.2 Hz, 3 x —CH₂CN), 1.70 (s, 2H, —NH₂). HRMS (ESI) m/z, C₁₃H₂₀N₄O₃ (M+H⁺). Theoretical value: 281.32, measured value: 281.20.

### Synthesis of Compound 10

Tri[(cyanoethoxy) methyl] aminomethane 9 (1.2 g, 4.28 mmol) was dissolved in 10 mL of

anhydrous ethanol, 2 mL concentrated sulfuric acid and 10 mL anhydrous ethanol were then added dropwise and slowly to the solution in a reaction flask at room temperature. The reaction solution was heated to 80° C. and kept at reflux state for about 36 hours. After cooling the reaction solution to room temperature, 25 mL of ice solution of saturated sodium bicarbonate was added. Ethanol was distilled by rotary evaporation under reduced pressure. The aqueous solution was extracted with ethyl acetate (2×50 mL), the obtained organic phase was dried with anhydrous sodium sulfate, and then subjected to rotary evaporation and concentration in reduced pressure to obtain a pale yellow oily substance 10 (0.8 g, 46%). This crude product was directly used in the next reaction without further purification.

### Synthesis of Compound 11

Crude product compound 10 (0.8 g, 1.9 mmol) was dissolved in 20 mL of dichloromethane. Di-tert-butyl dicarbonate (2 mL, 8.8 mmol) and 5 mL of triethylamine were added to the reaction solution. The reaction solution was stirred for 14 hours at room temperature. The reaction solution was poured into an aqueous solution containing saturated sodium bicarbonate, extracted with dichloromethane (2×50 mL), and the organic phase was subjected to washing with water. The organic phase was separated out, and dried with anhydrous sodium sulfate, and rotary evaporated and concentrated in reduced pressure to a semi-dry state, purification was further performed with a silica gel chromatographic column, a gradient elution was used, initially washed with dichloromethane solvent, then rinsed with a mixed solvent (containing dichloromethane/methanol, 96:4, v/v), the product components were collected, and the solvent was drained under reduced pressure to obtain a near-white oily substance 11 (0.5 g, 51%), ¹H NMR (CDCl₃): δ, 4.92 (b, 1H, —CONH—), 4.14 (m, 3x2H, —CO₂CH₂—), 3.69 (m, 3x2H,-OCH2-), 3.63 (s, 3x2H,-OCH2-), 2.53 (m, 3x2H, —COCH₂—), 1.45 (s, 3x3H, —CH₃), 1.26 (t, 3x3H, —CH₂CH₃). HRMS (ESI) m/z, C₂₄H₄₃NO₁₁ (M+H⁺). Theoretical value: 522.60, measured value: 522.40.

### Synthesis of Compound 12

Boc protected compound 11 (0.6 g, 1.43 mmol) was dissolved in 20 mL of absolute ethanol, 4 mL of sodium hydroxide solution (4M) was added dropwise and slowly into the reaction solution, the temperature of reaction solution was kept at 0° C. and stirred for 14 hours. The reaction progress was monitored constantly by LC-MS spectrum, while the peak of the reactant was vanished, the reaction solution was subjected to rotary evaporation under reduced pressure, after ethanol was evaporated, 10 mL of potassium bisulfate (1M) was added to the reaction solution and continuously stirred for 15 minutes at 0° C. The reaction solution was extracted with ethyl acetate (2×50 mL), the obtained organic phase was dried with anhydrous sodium sulfate, then rotary evaporated and concentrated in reduced pressure to obtain a viscous substance 12 (0.5 g, 81%). This crude product was directly used in the next step without further purification. 1H NMR (CDC13): d, 9.40 (b, 3H, —CO₂H), 5.0 (b, 1H, —CONH—), 3.70 (m, m, 3x2H, —OCH₂—), 3.65 (s, 3x2H, —OCH₂—), 2.60 (m, 3x2H, —COCH₂—), 1.42 (s, 3x3H, —CH₃). HRMS (ESI) m/z, C₁₈H₃₁NO₁₁ (M+H⁺). Theoretical values: 438.32, measured values: 438.20.

Compound 14 was prepared according to the following route:

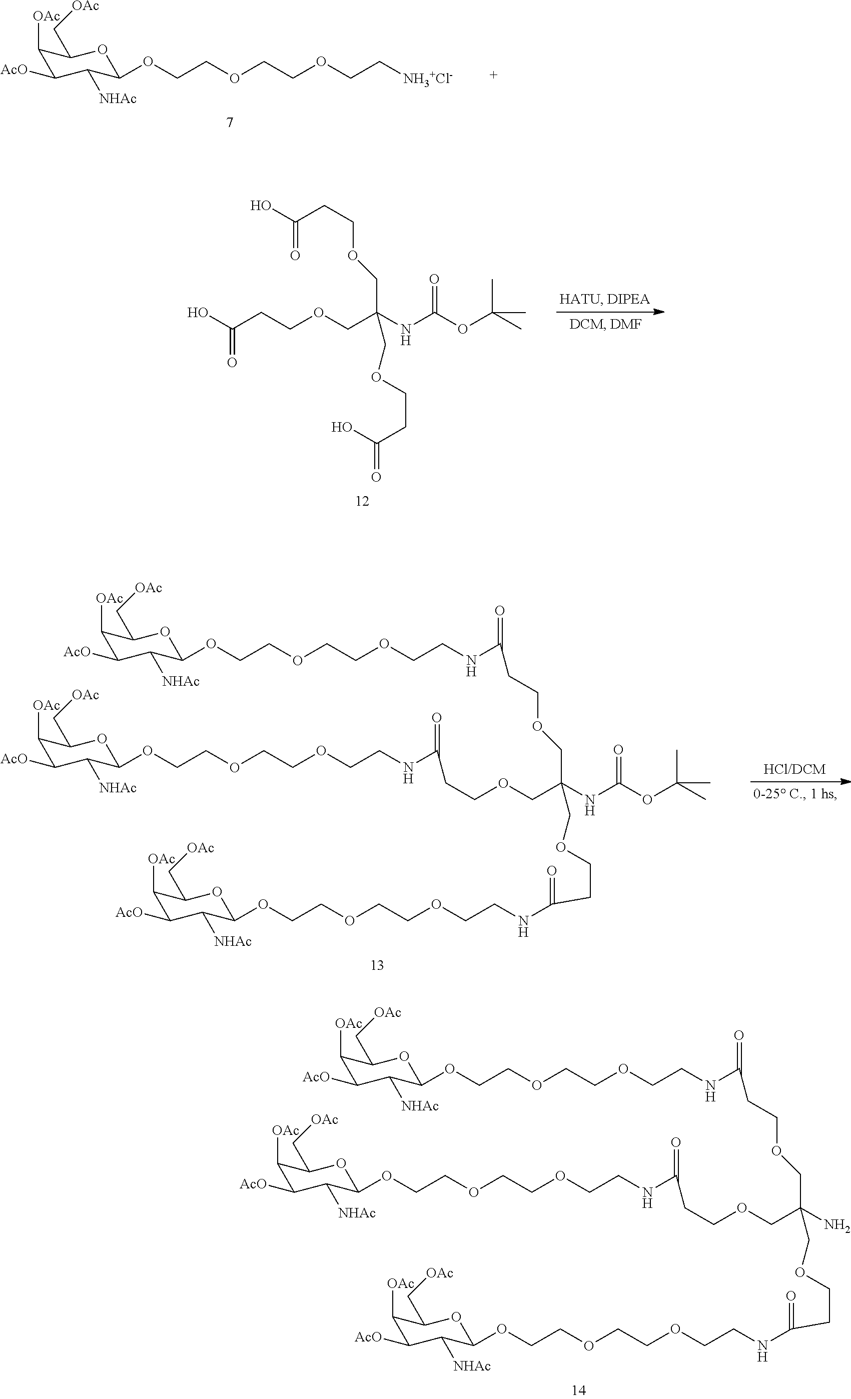

### Synthesis of Compound 13

Tricarboxylic acid 12 (0.5 g, 1.14 mmol) was dissolved in 20 mL of dichloromethane, 2-(7-azobenzotriazole)-N,N,N′,N′-tetramethylurea hexafluorophosphate (1.3 g, 3.42 mmol) and N,N-diisopropylethylamine (0.2 g, 3.95 mmol) were added, 8mL of dimethylformamide was added simultaneously. The compound 7 (2.19 g, 4.08 mmol) was dissolved in 5 mL of dimethylformamide, 10 and 1 mL of N,N-diisopropylethylamine was added. The two solutions were blended and stirred at room temperature and continuously stirred for 14 hours. The complete disappearance of the reactants was confirmed by the chromatographic detection. 20 mL of aqueous solution of saturated sodium bicarbonate was added into the reaction solution, and extracted with 2×50 mL of dichloromethane, the organic phase was rotary evaporated and concentrated to a semi-dry state, purification was further performed with a silica gel chromatographic column, a gradient elution was used, initially washed with dichloromethane solvent, then rinsed with a mixed solvent (containing dichloromethane/methanol, 85:15, v/v), the product components were collected, the solvent was drained under reduced pressure to obtain the near-yellow oily crude product 13 (2 g, 86%). This crude product 13 was further purified by using a reversed-phase chromatographic column, the rinsing solvent was a mixed solvent (containing H₂O/MeOH, 1:1, v/v), the product components were collected, then rotary evaporated and concentrated to a full dry state to obtain a compound 13 (1.24 g, 60%). ¹H NMR (CDCl₃): δ5.32 (d, 3H, J=3.0 Hz, sugar-H-4′), 5.18 (dd, 3H, sugar-H-3′), 4.78 (d, 3H, sugar-H-1′), 4.18-4.06 (m, 24H,-OCH2, sugar-H-5′), 3.93 (m, 9H, sugar-2xH-6′, sugar-H-2′), 3.77, 3.64, 3.46 (m, 6H, —CH₂NH—), 2.44, 2.20 (m, 6H, —COCH₂—), 2.15 (s, 9H, —NHCOCH₃), 2.09 (s, 2H, —CH₂—), 2.05, 1.99, 1.95 (3xs, 27H, —OCOCH₃), 1.81 (s, 9H, CH₃, Boc). HRMS (ESI) m/z, C₇₈H₁₂₇N₇O₄₁ (M+2H⁺)/2. Theoretical value: 910.1, measured value: 910.0.

### Synthesis of Compound 14

Purified Compound 13 (2 g, 1.1 mmol) was dissolved in 30 mL of dichloromethane, 1 mL of hydrochloric acid solution (4M) and 1 mL of dioxan were slowly added into the dichloromethane reaction solution at a temperature of 0° C. The reaction solution was stirred continuously for 30 minutes at a temperature of 0° C., and then stirred continuously for 30 minutes at room temperature.

The reactant was subjected to rotary evaporation and concentration to a full dry state to obtain a white foamy crude product 14 (1.7 g, 90%). This crude product was directly used in the next step without further purification. 1H NMR (CDCl₃): δ, 8.20 (b, 2H, —NH₂), 5.35 (d, 3H, J=3.0 Hz, sugar-H-4′), 5.22 (dd, 3H, sugar-H-3′), 4.80 (d, 3H, sugar-H-1′), 4.13 (m, 9H, sugar-2xH-6′, sugar-H-2′), 3.94-3.44 (m, 24H,-OCH₂, sugar-H-5′), 3.77, 3.64, 3.46 (m, 6H, —CH₂NH—), 2.55, 2.43 (m, 6H, —COCH₂—), 2.15 (s, 9H, —NHCOCH₃), 2.09 (s, 2H, —CH₂—), 2.05, 1.98, 1.96 (3xs, 27H, —OCOCH₃). HRMS (ESI) m/z, C₇₃H₁₁₉N₇O₃₉ (M+H⁺)/2. Theoretical value: 860.4, measured value: 860.0.

Compound 21 was prepared according to the following route:

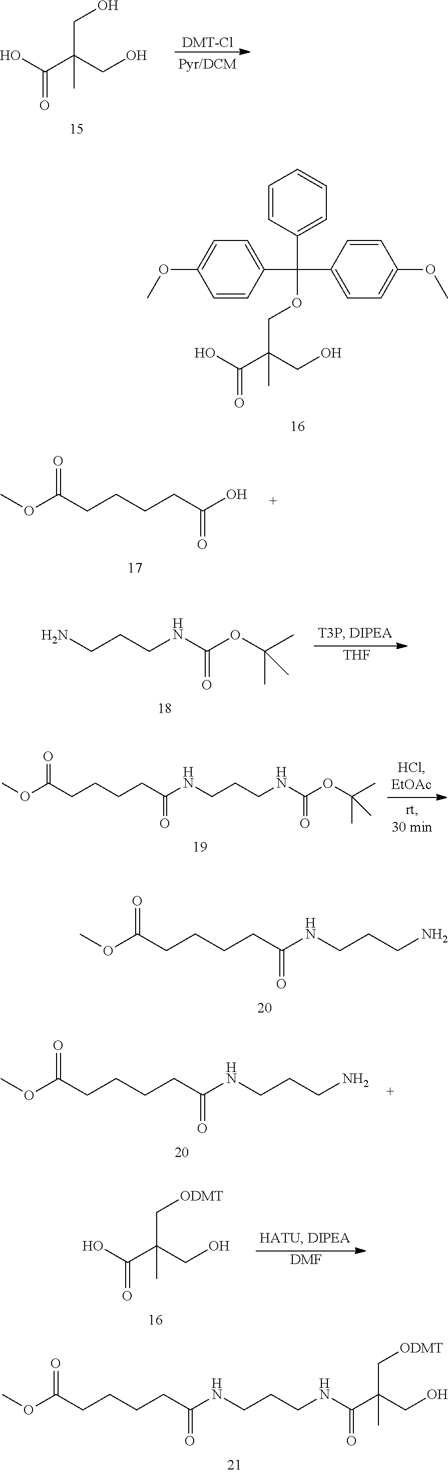

### Synthesis of Compound 16

4,4′-dimethoxytrityl chloride (1.8 g, 5.3 mmol) was dissolved in 5mL of dichloromethane, the solution was added dropwise and slowly into an anhydrous pyridine (10 mL) solution containing 3-hydroxy-2-hydroxymethyl-2-methyl-propanoic acid 15 (0.8 g, 5.97 mmol) at room temperature. The solution was stirred continuously for 14 hours at room temperature. 20 mL of water was added to the reaction mixture and extracted with 2×50 mL of ethyl acetate. The organic phase was subjected to rotary evaporation and concentration to a semi-dry state, purification was further performed with a silica gel chromatographic column, a gradient elution was used, initially washed with n-hexane solvent, and then eluted with a mixed solvent (containing n-hexane/ethyl acetate, 1:1, v/v), the product components were collected, the solvent was drained under reduced pressure to obtain a yellow solid 16 (1.5 g, 58%). The product 16 was directly used in the next step.

### Synthesis of Compound 19

Mono-methyl adipate 17 (0.16 g, 1 mmol) and N-(tert-butoxycarbonyl)-1,3-diaminopropane N-(3-aminopropyl) tert-butyl carbamate 18 (0.174 g, 1 mmol) were dissolved in 5 mL of anhydrous tetrahydrofuran at room temperature. The solution was blended with 0.892 mL of 1-propylphosphoric acid cyclic anhydride (0.892 mL, 1.5 mmol, in 50% (volume ratio 1:1) ethyl acetate) and 0.522 mL of N,N-diisopropylethylamine (DIPEA, 0.522 mL, 3 mmol). The mixed reaction solution was continuously stirred for 30 minutes at room temperature, 20 mL of ethyl acetate was then added into the reaction solution for dilution, and 20mL of saturated salt solution was added simultaneously, and extracted with 2×20mL of ethyl acetate. The organic phase was separated, and dried with anhydrous sodium sulfate, then rotary evaporated and concentrated to a full dry state, resulting in a yellowish foamed crude product 19 (0.29 g, 91%), which was directly used in the next step without further purification. ¹H NMR (CDCl₃), δ, 5.89 (b, 1H, —CONH—), 4.97 (b, 1H, —NHCO—), 3.75 (s, 3H, —CH₃), 3.27 (m, 2H), 3.15 (m, 2H), 2.34 (m, 2H), 2.19 (m, 2H), 1.67 (m 2x2H), 1.64 (m, 2H), 1.4 (s, 9H). HRMS (ESI) m/z, C₁₅H₂₈N₂O₅. Theoretical values: 316.39, measured values: 316.40.

### Synthesis of Compound 20

Compound 19 (0.8 g, 2.5 mmol) was dissolved in 5 mL of ethyl acetate, the reaction solution was mixed with 3.2 mL of aqueous hydrochloric acid solution (4M), and 5 mL of dioxane was further added. The mixed reaction solution was continuously stirred for 30 minutes at room temperature. After rotary evaporation and concentration, a viscous and thick crude product 20 (0.5 g, 92%) was obtained, which was directly used for the next step.

### Synthesis of Compound 21

The crude product compound 20 (0.252 g, 1 mmol) was dissolved in 5 ml of dimethylformamide, the reaction solution was blended with 3-O-4,4′-dimethoxytrityl-2-hydroxy-2-methylpropionic acid 16 (0.45 g, 0.9 mmol), and added 2-(7-azabenzotriazo)-N,N,N′,N′-tetramethylurea hexafluorophosphate (0.46 g, 1.2 mmol) and N,N-diisopropylethylamine (0.52 mL) at 0° C. and stirred for 20 minutes. The reaction solution was slowly heated to room temperature and continuously stirred for 14 hours. The reaction solution was blended with 20 mL of saturated sodium chloride solution, and extracted with 2×50 mL of ethyl acetate, the organic phase was dried with anhydrous sodium sulfate and then subjected to rotary evaporation and concentration in reduced pressure to obtain a crude product 21. Purification was further performed with a silica gel chromatographic column, a gradient elution was used, initially washed with ethyl acetate solvent, followed by rinsing with a mixed solvent (containing ethyl acetate/methanol, 90:10, v/v), the product components were collected, and the solvent was drained under reduced pressure to obtain a compound 21 (0.38 g, 61%). ¹H NMR (CDCl₃): δ, 8.01 (b, 1H, —CONH—), 7.26 (m, 4H, Trityl), 7.05 (m, 4H, Trityl), 6.67 (m, 4H, Trityl), 6.48 (b, 1H, —NHCO—), 5.25 (s, 3H,-OCH₃), 3.78 (s, 6H, 2x-OCH₃), 3.64 (s, 4H, 2X-CH₂—), 3.26 (m, 2H, —NHCH₂—), 3.17 (m, 2H, —CH₂NH—), 2.79 (m, 3H, —OCH₃), 2.31 (m, 2H), 2.18 (m, 2H), 1.65-1.56 (m, 6H), 1.25 (s, 3H). HRMS (ESI) m/z, C₃₆H₄₆N₂O₈, (M+Na⁺). Theoretical value: 657.34, measured value: 657.40.

Compound (603A) was prepared according to the following route:

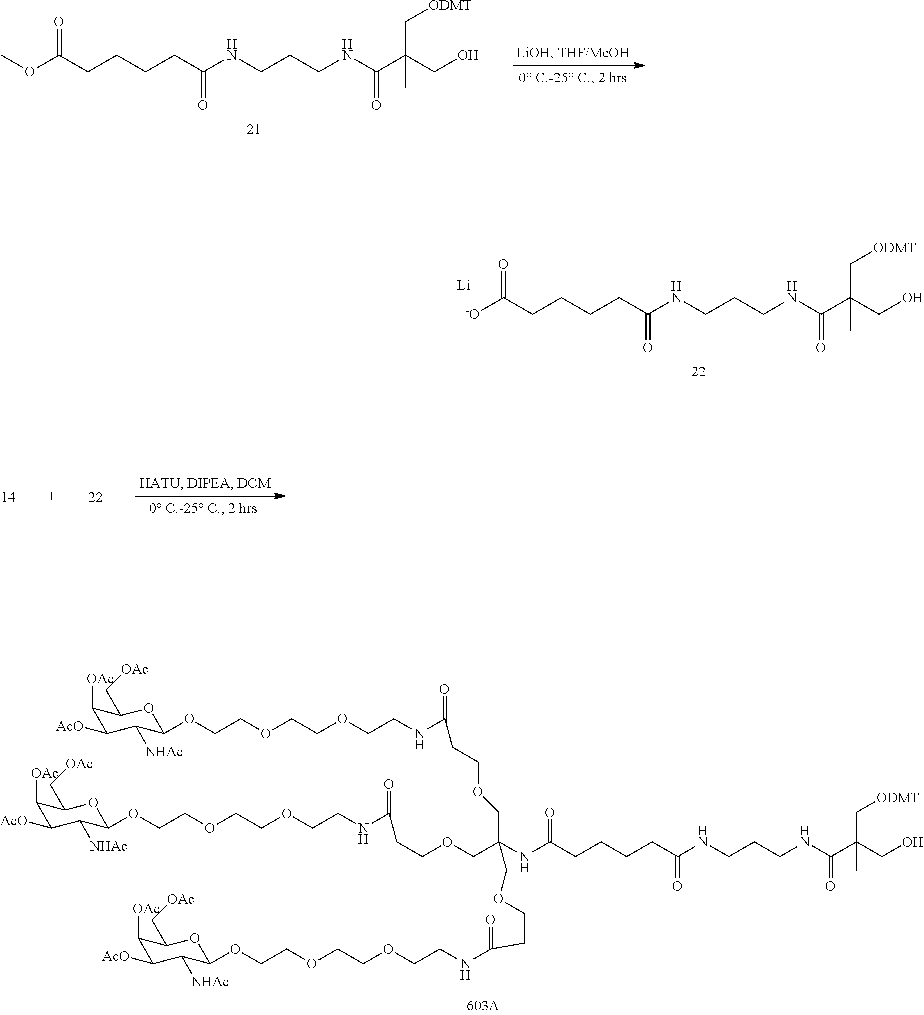

### Synthesis of Compound 22

The purified compound 21 (0.7 g, 1.1 mmol) was dissolved in 5 mL of anhydrous methanol, 1.5 mL of methanol solution of lithium chloride (2M) was added slowly into the reaction solution at the temperature of 0° C. The reaction solution was stirred at 0° C. for 30 min, the reaction solution was then heated to room temperature and continuously stirred for 2 hours at room temperature. After mixing the reaction solution with 2 mL of water at room temperature, the reaction solution was subjected to rotary evaporation and concentration to a semi-dry state such that the methanol was removed, and separation was performed by using the preparative reverse phase high pressure liquid chromatography, the mobile phase solvent was methanol and water (MeOH: H₂O, 1:1, v/v). The product components were collected, and the solvent was drained under reduced pressure to obtain a yellow solid compound 22 (0.66 g, 93%). ¹H NMR (CDCl₃): δ, 7.38 (m, 4H, Trityl), 7.29 (b, 1H, —CONH—), 7.28 (m, 5H, Trityl), 7.16 (b, 1H, —NHCO—), 6.79 (m, 4H, Trityl), 3.76 (s, 10H, 2x-OCH₃, 2x-CH₂), 3.71 (m, 2H, —CH₂—), 3.21 (m, 2H, —NHCH₂—), 2.07 (m, 2H, —CH₂NH—), 1.87 (m, 2H), 1.50 (m, 2H), 1.27 (m, 2H), 1.21 (s, 3H). HRMS (ESI) m/z, C35H43N208, (M+H⁺+Na⁺). Theoretical value: 643.72, measured value: 643.20.

Synthesis of Compound (603A)

The purified compound 22 (0.65 g, 1.04 mmol) was dissolved in 15 mL of dichloromethane, and mixed with 0.723 mL of N,N-diisopropylethylamine (4.16 mmol) at room temperature. The mixed solution was blended with the purified compound 14 (1.83 g, 1.04 mmol), 2-(7-azabenzotriazo)-N,N,N′,N′-tetramethylurea hexafluorophosphate (0.435 g, 1.1 mmol) and N,N-diisopropylethylamine (0.723 mL, 4.16 mmol) and stirred for 30 minutes under the temperature of 0° C. The reaction solution was further added with 1 mL of N,N-diisopropylethylamine and continuously stirred at 0° C. for 1 hour. The reaction temperature was gradually raised from 0° C. to room temperature, then continuously stirred for 2 hours. The reaction solution was blended with 5 mL of saturated sodium chloride solution, and extracted with 2×50 mL of dichloromethane, the organic phase was dried with anhydrous sodium sulfate, followed by rotary evaporation and concentration in reduced pressure to obtain a crude product 603A. Purification was further performed with a silica gel chromatographic column, a gradient elution was used, initially washed with dichloromethane solvent, then rinsed with a mixed solvent (containing dichloromethane/methanol/triethylamine, 94:5:1, v/v/v), the product components were collected, and the solvent was drained under reduced pressure to obtain a yellow solid compound 603A (1.56 g, 65%). ¹H NMR (CDCl₃): δ, 7.38 (m, 3H, —NH—), 7.28 (m, 4H, trityl), 7.26 (m, 1H, —NH—), 7.18 (m, 1H, —NH—), 6.84 (m, 5H, trityl), 6.37 (m, 4H, trityl), 5.33 (m, 3H, sugar-H-4′), 5.16 (dd, J=3.4 Hz, J=11.3 Hz, 3H, sugar-H-3′), 4.77 (d, J=8.4 Hz, 3H, sugar-H-1′), 4.18-4.07 (m, 3x2H, 3x1H, sugar-H-5′, sugar-H-6′), 3.94 (m, 3H, sugar-H-2′), 3.77-3.53 (m, 14H), 3.42 (m, 2H, —NHCH₂—), 3.30-3.19 (m, 2H, —CH₂NH—), 2.42 (m, 2H), 2.19 (m, 4H), 2.15 (s, 9H), 2.07 (m, 2H), 2.05 (s, 9H, 2.01 (s, 9H), 1.96 (s, 9H), 1.20 (s, 3H). HRMS (ESI) m/z, C₈₇H₁₄₃N₉O₄₄, (M-trityl+H+)/2. Theoretical value: 1,010.55, measured value: 1,010.4.

Conjugate (603B) triethylamine carboxylate was prepared according to the following route:

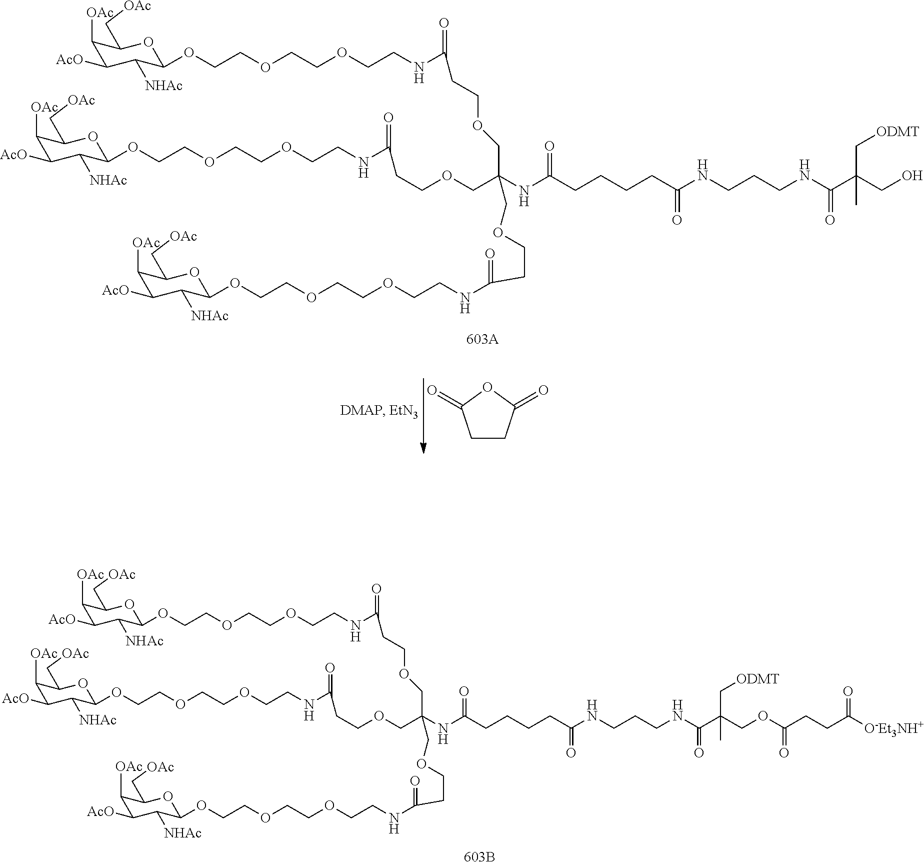

Compound (603A) (1.5 g, 0.646 mmol) was dissolved in 30 mL of dry dichloromethane, 5 mL of triethylamine was subsequently added. 4-dimethylaminopyridine (0.159 g, 1.3 mmol) was dissolved and stirred in the reaction solution, succinic anhydride (0.13 g, 1.3 mmol) was also dissolved in the reaction solution with the aid of stirring at room temperature, and the reaction was performed under stirring condition for 8 hours. Succinic anhydride (32 mg, 0.32 mmol) was further added and continuously stirred for 14 hours at room temperature. The reacted solution was then poured into a saturated saline solution, extracted with 2×50 mL of dichloromethane, the organic phase was separated, and dried with anhydrous sodium sulfate, then evaporated to a semi-dry state under reduced pressure. Purification was further performed with a silica gel chromatographic column, a gradient elution was used, initially washed with a mixed solvent (containing dichloromethane/methanol/triethylamine, 100:2:1, v/v/v), further rinsed with a mixed solvent (including dichloromethane/methanol/triethylamine, 100:5:1, v/v/v), then rinsed with a mixed solvent (containing dichloromethane/methanol/triethylamine, 100:5:1, v/v/v) to obtain the final product, the solvent was drained under the reduced pressure to obtain a white compound (603B) (1.56 g, 65%). ¹H NMR (CDCl₃) δ, ¹H NMR (CDCl₃): δ, 7.39 (m, 3H, —NH—), 7.28 (m, 5H, trityl), 7.22 (m, 1H, —NH—), 7.10 (m, 1H, —NH—), 6.80 (m, 4H, trityl), 6.37 (m, 4H, trityl), 5.32 (m, 3H, sugar-H-4′), 5.29 (s, 2H), 5.15 (dd, J-3.4Hz, J=11.3Hz, 3H, sugar-H-3′), 4.77 (d, J-8.4Hz, 3H, sugar-H-1′), 4.18-4.07 (m, 3x2H, 3x1H, sugar-H-5′, sugar-H-6′), 3.94 (m, 3H, sugar-H-2′), 3.67 (m, 9H), 3.61-3.53 (m, 42H), 3.42 (m, 2H, —NHCH₂—), 3.30-3.19 (m, 2H, —CH₂NH—), 2.42 (m, 2H), 2.19 (m, 4H), 2.15 (s, 9H), 2.07 (m, 2H), 2.05 (s, 9H, 2.01 (s, 9H), 1.96 (s, 9H), 1.23 (s, 3H). HRMS (ESI) m/z, C₁₁₂H₁₆₅N₉O₄₉, (M-H⁺)/2. Theoretical value: 1,209.27, measured value: 1,209.83.

Preparation of a conjugate (603C) by connecting the conjugate (603B) to the solid phase carrier according to the following process route:

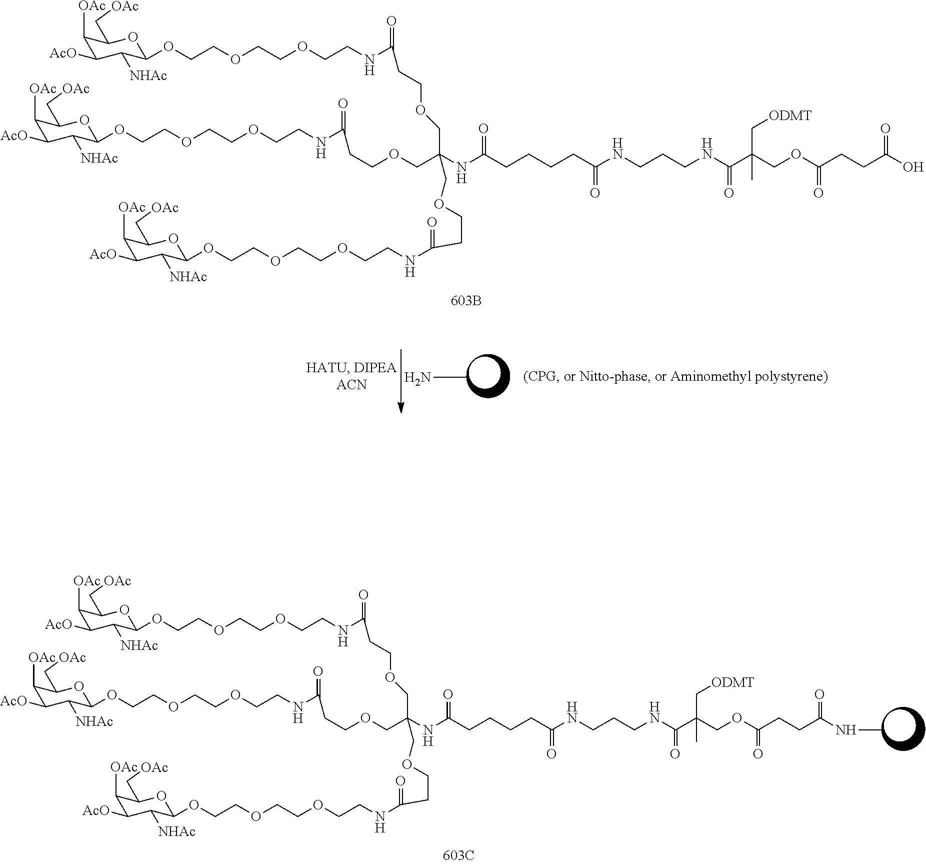

The conjugate (603B) (50 mg, 0.021mmol) and 2-(7-azabenzotriazo)-N,N,N′,N′-tetramethylurea hexafluorophosphate (10 mg, 0.026 mmol) were dissolved in 1.25 mL of anhydrous acetonitrile at room temperature. N,N-diisopropylethylamine (10 μL) was added into the reaction solution, after all reagents were dissolved, 125 mg of long-chain amino alkane glass sand (500° A, native lcaa-CPG, Chemgenes, USA) was added into the reaction solution. The solid-phase and liquid-phase were rotated and stirred for 300 rpm at room temperature. After the reaction lasted for 2 hours, the residue liquid was filtered, the long-chain amino alkane glass sand (solid phase carrier) was washed with acetonitrile for three times (3×1 mL). 0.5 mL of a tetrahydrofuran solution of capping reagent A (acetic anhydride) having a concentration of 10% (v/v) and 0.5 mL of capping reagent B (a mixed solution of N-methylimidazole, pyridine and acetonitrile at a concentration of 15:10:75, v/v/v) and the long-chain amino alkane glass sand were rotated and stirred for 1 hour at room temperature. The reaction solution was filtered, the long-chain amino alkane glass sand (solid phase carrier) was rinsed with acetonitrile for three times, then dried under reduced pressure for 2 hours by using an oil vacuum pump, a glass sand solid phase carrier (603C, 130 mg) was obtained.

The solid phase carrier 603C (8.3 mg) was weighed and added into 100 mL of a dichloromethane solution of trichloroacetic acid having a concentration of 3%, then rotated and stirred for 30 seconds and stood still for 1 minute. The supernatant was taken to measure the visible light absorption at 498 nm, its light absorbance was 0.309, a loading capacity of the conjugate 603C (i.e., a loading capacity of (603B) on CPG) was calculated as 53.25 μmol/g.

Preparation of sense and antisense strands of siRNA (preparation of siRNA drugs)

Pursuant to the solid phase synthesis method of phosphoramidites, the nucleoside monomers were linked one by one according to the sequence along the direction from 3′-5′ according to the above sequence. Each linking with a single nucleoside monomer included the four-step reaction of deprotection, coupling, capping and oxidation.

### Formulation of Solid Phase Synthesis Reagent:

The deprotection reagent was a dichloromethane solution of trichloroacetic acid (TCA) or dichloroacetic acid (DCA) having a concentration of 3% (v/v). The nucleoside monomers were dissolved in anhydrous acetonitrile having a concentration of 0.05-0.1M, which was added with a small amount of molecular sieve 3A° for anhydrous treatment. The coupling activator was anhydrous acetonitrile of 5-ethylthio-1H-tetrazole having a concentration of 0.25M or 0.45M, alternatively the activator may be selected from 1H-hetrazole, 5-benzyylthio-1H-tetrazole and 4,5-dicyanoimidazole. The capping reagent A was a tetrahydrofuran solution of acetic anhydride having a concentration of 10% (v/v). The capping reagent B was a mixed solvent of N-methylimidazole, pyridine and acetonitrile having a concentration of 15:10:75 (v/v/v). The oxidizing reagent was the water and pyridine solution of iodine having a concentration of 0.05M (95wt % of an aqueous pyridine solution). The sulfurization reagent was N-dimethylaminomethylidene)amino]-3H-1,2,4-dithiazoline-3-thione having a concentration of 0.05M (pyridine/acetonitrile, 2:3, v/v). The deprotection reagent was concentrated aqueous ammonia with a concentration of 28 wt. %.

Wherein the solid phase carrier for solid phase synthesis of nucleotides was commercially available and commonly used solid phase carrier (NittoPhase® HL UnyLinker™M 300; or 500° A, native lcaa-CPG, Chemgenes, USA).

### Steps of Solid Phase Synthesis:

A solid phase carrier was blended with a dichloromethane solution of trichloroacetic acid having a concentration of 3% (v/v) with a molar ratio of 1:30 on a synthesizer. The reaction was performed for 1.5 minutes at room temperature, and the operation was repeated for three times, the dropwise adding of a deprotection reagent was stopped when the color of elution solution of solid phase carrier was changed from red color to colorless. After repeated washing with anhydrous acetonitrile, the nucleoside monomer and activator (coupling activator, 5-ethylthio tetrazole) were added (at a molar ratio of nucleoside monomer to activator of 1:20), and a molar ratio of the solid phase carrier and nucleoside monomer were 1: (5-6). The reagents were at room temperature and the time of the solid phase reaction was 3-4 minutes for one cycle, and after two cycles, the reaction was stopped. After washing with anhydrous acetonitrile, the oxidizing reagent solution was added, a molar ratio of the solid phase carrier and the oxidizing reagent were 1:6. The reaction time of the oxidizing reagent and the solid phase carrier at room temperature was about 2 minutes, the operation was repeated twice. After the coupling reaction, if a sulfidation reaction step was required, the sulfidation reagent solution was added, a molar ratio of the solid phase carrier and the sulfidation reagent was 1:6. The reaction time of the sulfidation reagent and the solid phase carrier at room temperature was about 4-5 minutes, and the operation was repeated twice. The capping protection reaction was performed by adding the capping reagent, a molar ratio of the solid phase carrier and the capping reagent was 1:80. The reaction time of the capping reagent and the solid phase carrier at room temperature was about 1-2 minutes, and the operation was repeated twice. The aforementioned deprotection, coupling, oxidizing and capping steps were cycled until the coupling of the last nucleotide was completed. The solid phase carrier loaded with the sense strand or the antisense strand of the nucleic acid sequence was transferred into a vial bottle, an aqueous ammonia solution having a concentration of 28% was added, and the glass cap was screwed tightly for sealing, the base protecting group of the sense strand or the antisense strand was hydrolyzed and removed at the temperature of 55° C., while the sense strand or the antisense strand was hydrolyzed and separated from the solid phase carrier. The reaction was run for 16 hours. The resulting solution of the small nucleic acid strand was filtered and separated from the solid phase carrier. After concentration, a crude product of the small nucleic acid strand was obtained.

### Purification, Separation and Desalting by Using the Preparative High Pressure Liquid Chromatography

Small nucleic acid was purified by gradient elution of NaBr using a preparative type anion exchange chromatographic column (Source 15Q). Mobile phase A: 20 mM sodium phosphate (pH 8.0), mobile phase B: 20 mM sodium phosphate (pH 8.0), 1M sodium bromide in aqueous acetonitrile having a concentration of 10%. The column temperature was 65° C. The flow rate was 10 mL/min. The elution gradient was initiated a mobile phase A, followed by a change in mobile phase B from 0% to 20% at 12 minutes. The mobile phase B increased from 20% to 50% in the subsequent 15 minutes. The eluent was collected, and subjected to component analysis and component combination. Desalting was performed by using a reverse phase chromatography purification column, or a dialysis desalting process. The eluent was subjected to concentration and freeze drying to obtain purified small nucleotides. For the synthesized sense and antisense strands, purity was detected using the anion exchange liquid chromatogram (AEX-HPLC), and the full sequence molecular weight was identified and analyzed by reverse phase liquid chromatography- mass spectrometry (LC-MS), the measured value for molecular weight was consistent with the theoretical value to confirm success in synthesizing the nucleic acid sequence.

### Annealing

The synthesized sense strand (S strand) and antisense strand (AS strand) were blended at an equimolar ratio in a normal saline for injection, heated at a temperature of 90°° C. for 5 minutes, then cooled slowly to room temperature and stored in a refrigerator at 4° C. for 12 hours to form double-stranded structure through hydrogen bond, the siRNA drugs 30-34 were obtained (the sequences and modifications of nucleotides were shown in Table 3).

TABLE 3

| | Sense | Antisense |
|---|---|---|
| | strand | strand |
| | sequence | sequence |
| No. | (5′-3′) | (5′-3′) |
| 30 | GsCsUCGGUfGAfGUfGAUG | UsUfsCUfGCfCAUCACU |
| | GCAGAA-TriGalNAc (17) | CfACfCGAGCsUsU |
| | | (18 + UU) |
| 31 | GsGsUCACCfGAfCUfUCGA | AsCfsAUfUCfUCGAAGU |
| | GAAUGU-TriGalNAc (25) | CfGGfUGACCsUsU |
| | | (26 + UU) |
| 32 | CsCsCAAGCfAAfGCfAGAC | AsAfsAUfGUfCUGCUUf |
| | AUUU-TriGalNAc (31) | GCfUUGGGsUsU |
| | | (32 + UU) |
| 33 | GsCsAGGAAfCUfGAfGCCA | UsUfsUCfUGfGCUCAGf |
| | GAAA-TriGalNAc (37) | UUfCCUGCsUsU |
| | | (38 + UU) |
| 34 | GsCsCAAGCfCUfCUfUCUU | UsGfsAAfGUfAAGAAGA |
| | ACUUCA-TriGalNAc (45) | GfGCfUUGGCsUsU |
| | | (46 + UU) |

In the sequences shown in Table 3, the lowercase letter f indicates that the nucleotide adjacent to the left side of the letter f is a 2′-fluoro modified nucleotide (i.e., the 2′-OH of the pentose in the nucleotide is substituted by fluorine); the lowercase letter s indicates that two nucleotides adjacent to the left side and right side of the letter s are connected through a thiophosphate diester bond (i.e., the non-bridging oxygen atom in the phosphodiester bond is substituted by sulfur atom); the underlined nucleotide indicates that the 2′ hydroxyl group of the nucleotide is substituted by methoxyl group. TriGaNAc denotes a target group and a linkage group for target delivery (i.e., a moiety of formula (603) other than Nu).

The sequence and structure of the synthesized siRNA drugs were confirmed by LC-MS analysis data. The modified siRNA in Table 3 was provided with a more stable nucleic acid structure as well as high specificity and high affinity of base pairing. In addition, the nucleic acid having the above modification exhibited more excellent in vivo inhibitory effect, and can further reduce the in vivo immunogenicity of the nucleic acid of the present disclosure.

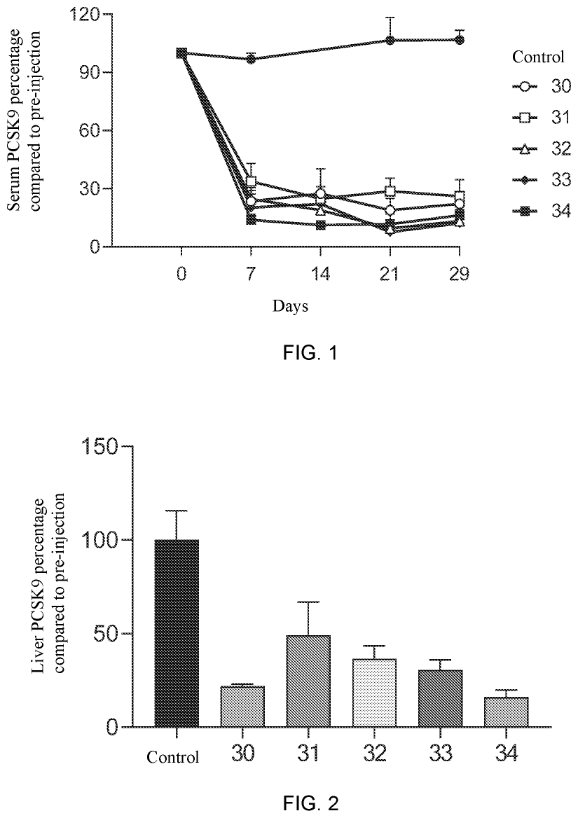

(II) The male humanized PCSK9 mice (4-6 weeks old, Shanghai Model Organisms Technology Co., Ltd.) were randomly grouped by weight, 3 mg/kg of siRNA drug (the dosage was based on siRNA alone) was injected into the mice on the zero day, the 2^{nd} day and the 4^{th} day, serum was taken on the zero day, the 7^{th} day, the 14th day, the 21^{st} day and the 29^{th} day, the PCSK9 concentration in serum was measured (control group was injected with normal saline). The serum human PCSK9 concentration measurement was accomplished by using a human PCSK9 ELISA kit (R&D Systems, Minneapolis, MN, USA, cat #DPC900), and the experimental procedures were performed as recommended by the manufacturer. FIG. 1 showed the effect that siRNA reduced the serum PCSK9 protein in humanized PCSK9 mice.

Meanwhile, the mice were killed at the 29^{th} day and the liver tissue was removed, RNA was extracted according to the operation instruction of RNAeasy Mini kit (QIAGEN, Article No. 74104). RT-PCR was performed according to the recommendation of High Capacity cDNA Reverse

Transcription Kits (Thermo Fisher, Article No. 4368814), 1 μg of RNA was contained in each reaction. The gene expression was quantified by using the real-time fluorescence PCR method, and the TaqMan probe of human PCSK9 was Hs00545399_m1, and the probe of the internal reference gene (mouse HPRT1) was Mm03024075_ml (Thermo Fisher Scientific, Waltham, MA, USA). The PCR conditions were 1 cycle for 20 sec at the temperature of 95° C., 40 cycles for 1 sec at 95° C. and 20 sec at 60° C., and the real-time fluorescence PCR instrument was StepOne Plus (Thermo Fisher). The PCSK9 gene expression was calculated based on the 2\hat{ }-ΔΔCt, and the mouse HPRTI gene expression was used as the internal reference. The expression level of PCSK9 gene in liver was expressed as a percentage of treated group vs. control group (Table 2). The male humanized PCSK9 mice (4-6 weeks old, Shanghai Model Organisms

Technology Co., Ltd.) were randomly grouped by weight, the siRNA drug 34 was injected into the mice on the zero day, serum was taken on the 14^{th} day, the PCSK9 concentration in serum was measured (the control group was the PCSK9 concentration of serum in mice injected with normal saline). The serum human PCSK9 concentration measurement was accomplished by using a human

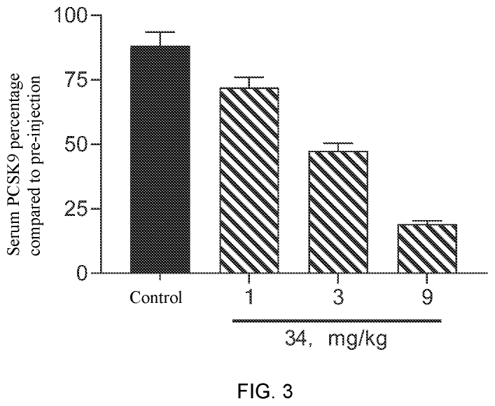

PCSK9 ELISA kit (R&D Systems, Minneapolis, MN, USA, cat #DPC900), and the experimental procedures were performed as recommended by the manufacturer. FIG. 3 showed that siRNA dose- dependently decreased the serum PCSK9 concentration of humanized PCSK9 mice.

All publications, patents and patent applications mentioned in the specification are incorporated herein by reference, the extent is same as that each individual publication, patent and patent application is specifically and individually incorporated herein by reference.

The above content describes in detail the preferred embodiments of the present disclosure, but the present disclosure is not limited thereto. A variety of simple modifications can be made in regard to the technical solutions of the present disclosure within the scope of the technical concept of the present disclosure, including a combination of individual technical features in any other suitable manner, such simple modifications and combinations thereof shall also be regarded as the content disclosed by the present disclosure, each of them falls into the protection scope of the present disclosure.

While this invention has been particularly shown and described with references to

preferred embodiments thereof, it will be understood by those skilled in the art that various changes in form and details may be made therein without departing from the scope of the invention encompassed by the appended claims.

1. A nucleic acid comprising a sense strand and an antisense strand, wherein the sense strand contains a sequence having at least 80% sequence identity to SEQ ID NO:1, SEQ ID NO:3, SEQ ID NO: 5, SEQ ID NO: 7, SEQ ID NO:9, SEQ ID NO: 11, SEQ ID NO: 13, SEQ ID NO: 15, SEQ ID NO: 17, SEQ ID NO:19, SEQ ID NO:21, SEQ ID NO:23, SEQ ID NO:25, SEQ ID NO:27, SEQ ID NO: 29, SEQ ID NO:31, SEQ ID NO:33, SEQ ID NO:35, SEQ ID NO:37, SEQ ID NO:39, SEQ ID NO: 41, SEQ ID NO:43, SEQ ID NO:45, SEQ ID NO:47, SEQ ID NO:49, SEQ ID NO:51, SEQ ID NO: 53, SEQ ID NO:55 or SEQ ID NO:57; the antisense strand contains a sequence having at least 80% sequence identity to SEQ ID NO:2, SEQ ID NO:4, SEQ ID NO:6, SEQ ID NO:8, SEQ ID NO: 10, SEQ ID NO: 12, SEQ ID NO:14, SEQ ID NO:16, SEQ ID NO:18, SEQ ID NO:20, SEQ ID NO: 22, SEQ ID NO:24, SEQ ID NO:26, SEQ ID NO:28, SEQ ID NO:30, SEQ ID NO:32, SEQ ID NO: 34, SEQ ID NO:36, SEQ ID NO:38, SEQ ID NO:40, SEQ ID NO:42, SEQ ID NO:44, SEQ ID NO: 46, SEQ ID NO:48, SEQ ID NO:50, SEQ ID NO:52, SEQ ID NO:54, SEQ ID NO:56 or SEQ ID NO: 58.

2. The nucleic acid of claim 1, wherein at least 80% sequence identity refers to that there is one different base between the sequences; the different base in the sense strand is the last base of the sense strand, the different base in the antisense strand is the first base of the antisense strand.

3. The nucleic acid of claim 1, wherein the sense strand has 16-30 nucleotides; and/or the antisense strand has 16-30 nucleotides.

4. The nucleic acid of claim 1, wherein the nucleic acid is at least one selected from siRNA-1 having a sense strand sequence of SEQ ID NO: 1 and an antisense strand sequence of SEQ ID NO:2, siRNA-2 having a sense strand sequence of SEQ ID NO:3 and an antisense strand sequence of SEQ ID NO: 4, siRNA-3 having a sense strand sequence of SEQ ID NO:5 and an antisense strand sequence of SEQ ID NO:6, siRNA-4 having a sense strand sequence of SEQ ID NO:7 and an antisense strand sequence of SEQ ID NO:8, siRNA-5 having a sense strand sequence of SEQ ID NO: 9 and an antisense strand sequence of SEQ ID NO: 10, siRNA-6 having a sense strand sequence of SEQ ID NO: 11 and an antisense strand sequence of SEQ ID NO: 12, siRNA-7 having a sense strand sequence of SEQ ID NO: 13 and an antisense strand sequence of SEQ ID NO:14, siRNA-8 having a sense strand sequence of SEQ ID NO:15 and an antisense strand sequence of SEQ ID NO: 16, siRNA-9 having a sense strand sequence of SEQ ID NO:17 and an antisense strand sequence of SEQ ID NO: 18, siRNA-10 having a sense strand sequence of SEQ ID NO: 19 and an antisense strand sequence of SEQ ID NO:20, siRNA-11 having a sense strand sequence of SEQ ID NO: 21 and an antisense strand sequence of SEQ ID NO:22, siRNA-12 having a sense strand sequence of SEQ ID NO:23 and an antisense strand sequence of SEQ ID NO:24, siRNA-13 having a sense strand sequence of SEQ ID NO:25 and an antisense strand sequence of SEQ ID NO:26, siRNA-14 having a sense strand sequence of SEQ ID NO:27 and an antisense strand sequence of SEQ ID NO:28, siRNA-15 having a sense strand sequence of SEQ ID NO:29 and an antisense strand sequence of SEQ ID NO:30, siRNA-16 having a sense strand sequence of SEQ ID NO:31 and an antisense strand sequence of SEQ ID NO:32, siRNA-17 having a sense strand sequence of SEQ ID NO:33 and an antisense strand sequence of SEQ ID NO:34, siRNA-18 having a sense strand sequence of SEQ ID NO:35 and an antisense strand sequence of SEQ ID NO:36, siRNA-19 having a sense strand sequence of SEQ ID NO:37 and an antisense strand sequence of SEQ ID NO: 38, siRNA-20 having a sense strand sequence of SEQ ID NO:39 and an antisense strand sequence of SEQ ID NO:40, siRNA-21 having a sense strand sequence of SEQ ID NO:41 and an antisense strand sequence of SEQ ID NO:42, siRNA-22 having a sense strand sequence of SEQ ID NO: 43 and an antisense strand sequence of SEQ ID NO:44, siRNA-23 having a sense strand sequence of SEQ ID NO:45 and an antisense strand sequence of SEQ ID NO:46, siRNA-24 having a sense strand sequence of SEQ ID NO:47 and an antisense strand sequence of SEQ ID NO:48, siRNA-25 having a sense strand sequence of SEQ ID NO:49 and an antisense strand sequence of SEQ ID NO:50, siRNA-26 having a sense strand sequence of SEQ ID NO:51 and an antisense strand sequence of SEQ ID NO:52, siRNA-27 having a sense strand sequence of SEQ ID NO:53 and an antisense strand sequence of SEQ ID NO:54, siRNA-28 having a sense strand sequence of SEQ ID NO:55 and an antisense strand sequence of SEQ ID NO:56, siRNA-29 having a sense strand sequence of SEQ ID NO:57 and an antisense strand sequence of SEQ ID NO: 58.

5. The nucleic acid of claim 1, wherein the nucleic acid is selected from a siRNA-23 having a sense strand sequence of SEQ ID NO: 45 and an antisense strand sequence of SEQ ID NO: 46, and having the modifications at the nucleotide as follows:
5′-GsCsCAAGCfCUfCUfUCUUACUUCA-3′
5′-UsGfsAAfGUfAAGAAGAGfGCfUUGGCsUsU-3′
wherein the lowercase letter f indicates that the nucleotide adjacent to the left side of the letter f is a 2′-fluoro modified nucleotide; the lowercase letter s indicates that two nucleotides adjacent to the left side and right side of the letter s are connected through a thiophosphate diester bond; the underlined nucleotide indicates that the 2′ hydroxyl group of the nucleotide is substituted by methoxyl group.

6. A targeted drug delivery system comprising a target group, a linkage group, and the nucleic acid of claim 1 connected with the target group via the linkage group.

7. The targeted drug delivery system of claim 6, wherein the targeted drug delivery system has a structure as shown below:

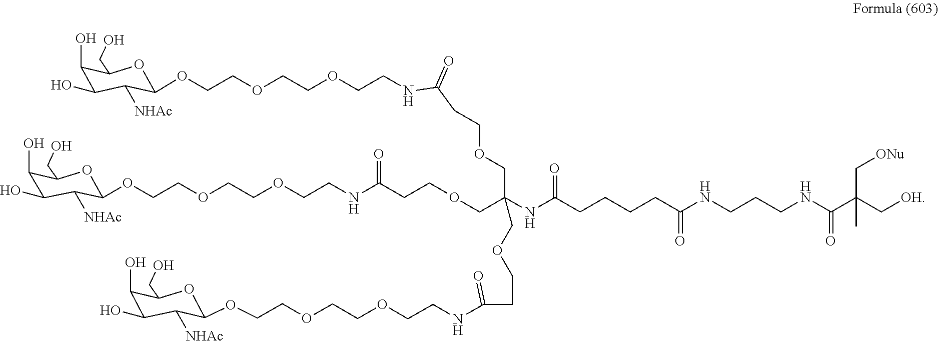

8. A pharmaceutical composition comprising the nucleic acid of claim 1, or the targeted drug delivery system of claim 6, and a pharmaceutically acceptable carrier.

9. A method of inhibiting PCSK9 comprising administering the nucleic acid of claim 1 to a patient suffering from the hyperlipidemia related diseases.

10. The method of claim 9, wherein the hyperlipidemia related diseases is at least one selected from coronary atherosclerosis heart disease, ischemic stroke, arteriosclerosis cerebral infarction, hypertension, diabetes mellitus, nephropathy and peripheral vascular diseases.

11. The method of claim 9, wherein the patient is a human patient.


SEQUENCE LISTING (59 sequences)

SEQ ID NO: 1 - DNA, 21 bp, synthetic construct
  source 1..21: mol_type = other DNA
  modified_base 4: mod_base = OTHER; note = uracil
  modified_base 8: mod_base = OTHER; note = uracil
  modified_base 12: mod_base = OTHER; note = uracil
  modified_base 15: mod_base = OTHER; note = uracil
agctcggtgagtgatggcaga

SEQ ID NO: 2 - DNA, 21 bp, synthetic construct
  source 1..21: mol_type = other DNA
  modified_base 1: mod_base = OTHER; note = uracil
  modified_base 3: mod_base = OTHER; note = uracil
  modified_base 8: mod_base = OTHER; note = uracil
  modified_base 12: mod_base = OTHER; note = uracil
  modified_base 21: mod_base = OTHER; note = uracil
tctgccatcactcaccgagct

SEQ ID NO: 3 - DNA, 19 bp, synthetic construct
  source 1..19: mol_type = other DNA
  modified_base 3: mod_base = OTHER; note = uracil
  modified_base 7: mod_base = OTHER; note = uracil
  modified_base 11: mod_base = OTHER; note = uracil
  modified_base 14: mod_base = OTHER; note = uracil
gctcggtgagtgatggcag

SEQ ID NO: 4 - DNA, 19 bp, synthetic construct
  source 1..19: mol_type = other DNA
  modified_base 2: mod_base = OTHER; note = uracil
  modified_base 7: mod_base = OTHER; note = uracil
  modified_base 11: mod_base = OTHER; note = uracil
ctgccatcactcaccgagc

SEQ ID NO: 5 - DNA, 19 bp, synthetic construct
  source 1..19: mol_type = other DNA
  modified_base 3: mod_base = OTHER; note = uracil
  modified_base 18: mod_base = OTHER; note = uracil
gctcacacagcaggaactg

SEQ ID NO: 6 - DNA, 19 bp, synthetic construct
  source 1..19: mol_type = other DNA
  modified_base 4..5: mod_base = OTHER; note = uracil
  modified_base 8: mod_base = OTHER; note = uracil
  modified_base 11: mod_base = OTHER; note = uracil
  modified_base 13: mod_base = OTHER; note = uracil
  modified_base 15: mod_base = OTHER; note = uracil
cagttcctgctgtgtgagc

SEQ ID NO: 7 - DNA, 19 bp, synthetic construct
  source 1..19: mol_type = other DNA
  modified_base 3: mod_base = OTHER; note = uracil
  modified_base 13: mod_base = OTHER; note = uracil
  modified_base 18: mod_base = OTHER; note = uracil
agtcaaggagcatggaatc

SEQ ID NO: 8 - DNA, 19 bp, synthetic construct
  source 1..19: mol_type = other DNA
  modified_base 3..4: mod_base = OTHER; note = uracil
  modified_base 8: mod_base = OTHER; note = uracil
  modified_base 11: mod_base = OTHER; note = uracil
  modified_base 14..15: mod_base = OTHER; note = uracil
  modified_base 19: mod_base = OTHER; note = uracil
gattccatgctccttgact

SEQ ID NO: 9 - DNA, 19 bp, synthetic construct
  source 1..19: mol_type = other DNA
  modified_base 1: mod_base = OTHER; note = uracil
  modified_base 4: mod_base = OTHER; note = uracil
  modified_base 9: mod_base = OTHER; note = uracil
  modified_base 11: mod_base = OTHER; note = uracil
tcctcaggtctccaccaag

SEQ ID NO: 10 - DNA, 19 bp, synthetic construct
  source 1..19: mol_type = other DNA
  modified_base 2..3: mod_base = OTHER; note = uracil
  modified_base 6: mod_base = OTHER; note = uracil
  modified_base 14: mod_base = OTHER; note = uracil
cttggtggagacctgagga

SEQ ID NO: 11 - DNA, 19 bp, synthetic construct
  source 1..19: mol_type = other DNA
  modified_base 1: mod_base = OTHER; note = uracil
  modified_base 8..9: mod_base = OTHER; note = uracil
  modified_base 13..15: mod_base = OTHER; note = uracil
  modified_base 17: mod_base = OTHER; note = uracil
tggaggcttagctttctgg

SEQ ID NO: 12 - DNA, 19 bp, synthetic construct
  source 1..19: mol_type = other DNA
  modified_base 10: mod_base = OTHER; note = uracil
  modified_base 16: mod_base = OTHER; note = uracil
ccagaaagctaagcctcca

SEQ ID NO: 13 - DNA, 19 bp, synthetic construct
  source 1..19: mol_type = other DNA
  modified_base 2: mod_base = OTHER; note = uracil
  modified_base 19: mod_base = OTHER; note = uracil
ctgagccagaaacgcagat

SEQ ID NO: 14 - DNA, 19 bp, synthetic construct
  source 1..19: mol_type = other DNA
  modified_base 2: mod_base = OTHER; note = uracil
  modified_base 4: mod_base = OTHER; note = uracil
  modified_base 8..10: mod_base = OTHER; note = uracil
  modified_base 12: mod_base = OTHER; note = uracil
  modified_base 16: mod_base = OTHER; note = uracil
atctgcgtttctggctcag

SEQ ID NO: 15 - DNA, 19 bp, synthetic construct
  source 1..19: mol_type = other DNA
  modified_base 2: mod_base = OTHER; note = uracil
  modified_base 9: mod_base = OTHER; note = uracil
  modified_base 19: mod_base = OTHER; note = uracil
ctgcagcgtccacacagct

SEQ ID NO: 16 - DNA, 19 bp, synthetic construct
  source 1..19: mol_type = other DNA
  modified_base 4: mod_base = OTHER; note = uracil
  modified_base 6: mod_base = OTHER; note = uracil
  modified_base 8: mod_base = OTHER; note = uracil
  modified_base 15: mod_base = OTHER; note = uracil
agctgtgtggacgctgcag

SEQ ID NO: 17 - DNA, 21 bp, synthetic construct
  source 1..21: mol_type = other DNA
  modified_base 3: mod_base = OTHER; note = uracil
  modified_base 7: mod_base = OTHER; note = uracil
  modified_base 11: mod_base = OTHER; note = uracil
  modified_base 14: mod_base = OTHER; note = uracil
gctcggtgagtgatggcagaa

SEQ ID NO: 18 - DNA, 21 bp, synthetic construct
  source 1..21: mol_type = other DNA
  modified_base 1..2: mod_base = OTHER; note = uracil
  modified_base 4: mod_base = OTHER; note = uracil
  modified_base 9: mod_base = OTHER; note = uracil
  modified_base 13: mod_base = OTHER; note = uracil
ttctgccatcactcaccgagc

SEQ ID NO: 19 - DNA, 19 bp, synthetic construct
  source 1..19: mol_type = other DNA
  modified_base 4: mod_base = OTHER; note = uracil
  modified_base 6: mod_base = OTHER; note = uracil
  modified_base 8..9: mod_base = OTHER; note = uracil
  modified_base 13: mod_base = OTHER; note = uracil
  modified_base 18..19: mod_base = OTHER; note = uracil
gcatgtcttccatggcctt

SEQ ID NO: 20 - DNA, 19 bp, synthetic construct
  source 1..19: mol_type = other DNA
  modified_base 8: mod_base = OTHER; note = uracil
  modified_base 17: mod_base = OTHER; note = uracil
aaggccatggaagacatgc

SEQ ID NO: 21 - DNA, 19 bp, synthetic construct
  source 1..19: mol_type = other DNA
  modified_base 3: mod_base = OTHER; note = uracil
  modified_base 9: mod_base = OTHER; note = uracil
  modified_base 11: mod_base = OTHER; note = uracil
  modified_base 13: mod_base = OTHER; note = uracil
  modified_base 15: mod_base = OTHER; note = uracil
  modified_base 18: mod_base = OTHER; note = uracil
ggtggaggtgtatctccta

SEQ ID NO: 22 - DNA, 19 bp, synthetic construct
  source 1..19: mol_type = other DNA
  modified_base 1: mod_base = OTHER; note = uracil
  modified_base 8: mod_base = OTHER; note = uracil
  modified_base 14: mod_base = OTHER; note = uracil
taggagatacacctccacc

SEQ ID NO: 23 - DNA, 19 bp, synthetic construct
  source 1..19: mol_type = other DNA
  modified_base 3: mod_base = OTHER; note = uracil
  modified_base 11..12: mod_base = OTHER; note = uracil
  modified_base 19: mod_base = OTHER; note = uracil
ggtcaccgacttcgagaat

SEQ ID NO: 24 - DNA, 19 bp, synthetic construct
  source 1..19: mol_type = other DNA
  modified_base 2..3: mod_base = OTHER; note = uracil
  modified_base 5: mod_base = OTHER; note = uracil
  modified_base 11: mod_base = OTHER; note = uracil
  modified_base 15: mod_base = OTHER; note = uracil
attctcgaagtcggtgacc

SEQ ID NO: 25 - DNA, 21 bp, synthetic construct
  source 1..21: mol_type = other DNA
  modified_base 3: mod_base = OTHER; note = uracil
  modified_base 11..12: mod_base = OTHER; note = uracil
  modified_base 19: mod_base = OTHER; note = uracil
  modified_base 21: mod_base = OTHER; note = uracil
ggtcaccgacttcgagaatgt

SEQ ID NO: 26 - DNA, 21 bp, synthetic construct
  source 1..21: mol_type = other DNA
  modified_base 4..5: mod_base = OTHER; note = uracil
  modified_base 7: mod_base = OTHER; note = uracil
  modified_base 13: mod_base = OTHER; note = uracil
  modified_base 17: mod_base = OTHER; note = uracil
acattctcgaagtcggtgacc

SEQ ID NO: 27 - DNA, 21 bp, synthetic construct
  source 1..21: mol_type = other DNA
  modified_base 6: mod_base = OTHER; note = uracil
  modified_base 11: mod_base = OTHER; note = uracil
  modified_base 17: mod_base = OTHER; note = uracil
  modified_base 21: mod_base = OTHER; note = uracil
agccatcacctaggactgact

SEQ ID NO: 28 - DNA, 21 bp, synthetic construct
  source 1..21: mol_type = other DNA
  modified_base 3: mod_base = OTHER; note = uracil
  modified_base 7: mod_base = OTHER; note = uracil
  modified_base 10: mod_base = OTHER; note = uracil
  modified_base 14: mod_base = OTHER; note = uracil
  modified_base 17: mod_base = OTHER; note = uracil
  modified_base 21: mod_base = OTHER; note = uracil
agtcagtcctaggtgatggct

SEQ ID NO: 29 - DNA, 21 bp, synthetic construct
  source 1..21: mol_type = other DNA
  modified_base 4..6: mod_base = OTHER; note = uracil
  modified_base 12..13: mod_base = OTHER; note = uracil
gcatttcaccattcaaacagg

SEQ ID NO: 30 - DNA, 21 bp, synthetic construct
  source 1..21: mol_type = other DNA
  modified_base 3: mod_base = OTHER; note = uracil
  modified_base 5..7: mod_base = OTHER; note = uracil
  modified_base 11: mod_base = OTHER; note = uracil
  modified_base 14: mod_base = OTHER; note = uracil
  modified_base 19: mod_base = OTHER; note = uracil
cctgtttgaatggtgaaatgc

SEQ ID NO: 31 - DNA, 19 bp, synthetic construct
  source 1..19: mol_type = other DNA
  modified_base 17..19: mod_base = OTHER; note = uracil
cccaagcaagcagacattt

SEQ ID NO: 32 - DNA, 19 bp, synthetic construct
  source 1..19: mol_type = other DNA
  modified_base 4: mod_base = OTHER; note = uracil
  modified_base 6: mod_base = OTHER; note = uracil
  modified_base 8: mod_base = OTHER; note = uracil
  modified_base 11..12: mod_base = OTHER; note = uracil
  modified_base 15..16: mod_base = OTHER; note = uracil
aaatgtctgcttgcttggg

SEQ ID NO: 33 - DNA, 19 bp, synthetic construct
  source 1..19: mol_type = other DNA
  modified_base 9: mod_base = OTHER; note = uracil
  modified_base 11: mod_base = OTHER; note = uracil
  modified_base 15: mod_base = OTHER; note = uracil
  modified_base 18..19: mod_base = OTHER; note = uracil
ccaacaactgtccctcctt

SEQ ID NO: 34 - DNA, 19 bp, synthetic construct
  source 1..19: mol_type = other DNA
  modified_base 13..14: mod_base = OTHER; note = uracil
  modified_base 16..17: mod_base = OTHER; note = uracil
aaggagggacagttgttgg

SEQ ID NO: 35 - DNA, 19 bp, synthetic construct
  source 1..19: mol_type = other DNA
  modified_base 6: mod_base = OTHER; note = uracil
  modified_base 9..10: mod_base = OTHER; note = uracil
  modified_base 12: mod_base = OTHER; note = uracil
  modified_base 19: mod_base = OTHER; note = uracil
ggagatgcttctaaggcat

SEQ ID NO: 36 - DNA, 19 bp, synthetic construct
  source 1..19: mol_type = other DNA
  modified_base 2: mod_base = OTHER; note = uracil
  modified_base 6..7: mod_base = OTHER; note = uracil
  modified_base 15: mod_base = OTHER; note = uracil
  modified_base 17: mod_base = OTHER; note = uracil
atgccttagaagcatctcc

SEQ ID NO: 37 - DNA, 19 bp, synthetic construct
  source 1..19: mol_type = other DNA
  modified_base 9: mod_base = OTHER; note = uracil
gcaggaactgagccagaaa

SEQ ID NO: 38 - DNA, 19 bp, synthetic construct
  source 1..19: mol_type = other DNA
  modified_base 1..3: mod_base = OTHER; note = uracil
  modified_base 5: mod_base = OTHER; note = uracil
  modified_base 9: mod_base = OTHER; note = uracil
  modified_base 13..14: mod_base = OTHER; note = uracil
  modified_base 17: mod_base = OTHER; note = uracil
tttctggctcagttcctgc

SEQ ID NO: 39 - DNA, 19 bp, synthetic construct
  source 1..19: mol_type = other DNA
  modified_base 3..5: mod_base = OTHER; note = uracil
  modified_base 7: mod_base = OTHER; note = uracil
  modified_base 11: mod_base = OTHER; note = uracil
  modified_base 16: mod_base = OTHER; note = uracil
  modified_base 18: mod_base = OTHER; note = uracil
gctttctggatggcatcta

SEQ ID NO: 40 - DNA, 19 bp, synthetic construct
  source 1..19: mol_type = other DNA
  modified_base 1: mod_base = OTHER; note = uracil
  modified_base 5: mod_base = OTHER; note = uracil
  modified_base 10: mod_base = OTHER; note = uracil
tagatgccatccagaaagc

SEQ ID NO: 41 - DNA, 19 bp, synthetic construct
  source 1..19: mol_type = other DNA
  modified_base 4: mod_base = OTHER; note = uracil
  modified_base 7..8: mod_base = OTHER; note = uracil
  modified_base 11: mod_base = OTHER; note = uracil
  modified_base 18..19: mod_base = OTHER; note = uracil
ccctgattaatggaggctt

SEQ ID NO: 42 - DNA, 19 bp, synthetic construct
  source 1..19: mol_type = other DNA
  modified_base 6: mod_base = OTHER; note = uracil
  modified_base 10..11: mod_base = OTHER; note = uracil
  modified_base 14: mod_base = OTHER; note = uracil
aagcctccattaatcaggg

SEQ ID NO: 43 - DNA, 19 bp, synthetic construct
  source 1..19: mol_type = other DNA
  modified_base 15..16: mod_base = OTHER; note = uracil
  modified_base 18..19: mod_base = OTHER; note = uracil
ccaaggaggcaggattctt

SEQ ID NO: 44 - DNA, 19 bp, synthetic construct
  source 1..19: mol_type = other DNA
  modified_base 6: mod_base = OTHER; note = uracil
  modified_base 9: mod_base = OTHER; note = uracil
  modified_base 13: mod_base = OTHER; note = uracil
  modified_base 16..17: mod_base = OTHER; note = uracil
aagaatcctgcctccttgg

SEQ ID NO: 45 - DNA, 21 bp, synthetic construct
  source 1..21: mol_type = other DNA
  modified_base 9: mod_base = OTHER; note = uracil
  modified_base 11..12: mod_base = OTHER; note = uracil
  modified_base 14..15: mod_base = OTHER; note = uracil
  modified_base 18..19: mod_base = OTHER; note = uracil
gccaagcctcttcttacttca

SEQ ID NO: 46 - DNA, 21 bp, synthetic construct
  source 1..21: mol_type = other DNA
  modified_base 1: mod_base = OTHER; note = uracil
  modified_base 6: mod_base = OTHER; note = uracil
  modified_base 17..18: mod_base = OTHER; note = uracil
tgaagtaagaagaggcttggc

SEQ ID NO: 47 - DNA, 21 bp, synthetic construct
  source 1..21: mol_type = other DNA
  modified_base 6..8: mod_base = OTHER; note = uracil
  modified_base 14..15: mod_base = OTHER; note = uracil
gggcatttcaccattcaaaca

SEQ ID NO: 48 - DNA, 21 bp, synthetic construct
  source 1..21: mol_type = other DNA
  modified_base 3: mod_base = OTHER; note = uracil
  modified_base 9: mod_base = OTHER; note = uracil
  modified_base 13: mod_base = OTHER; note = uracil
  modified_base 18..20: mod_base = OTHER; note = uracil
agtaaaggtggctcaggtttc

SEQ ID NO: 49 - DNA, 21 bp, synthetic construct
  source 1..21: mol_type = other DNA
  modified_base 7..9: mod_base = OTHER; note = uracil
  modified_base 12: mod_base = OTHER; note = uracil
  modified_base 14: mod_base = OTHER; note = uracil
  modified_base 17: mod_base = OTHER; note = uracil
  modified_base 19: mod_base = OTHER; note = uracil
  modified_base 21: mod_base = OTHER; note = uracil
gccacctttactctgctctat

SEQ ID NO: 50 - DNA, 21 bp, synthetic construct
  source 1..21: mol_type = other DNA
  modified_base 2: mod_base = OTHER; note = uracil
  modified_base 12: mod_base = OTHER; note = uracil
  modified_base 18: mod_base = OTHER; note = uracil
atagagcagagtaaaggtggc

SEQ ID NO: 51 - DNA, 19 bp, synthetic construct
  source 1..19: mol_type = other DNA
  modified_base 12..14: mod_base = OTHER; note = uracil
  modified_base 16: mod_base = OTHER; note = uracil
  modified_base 18..19: mod_base = OTHER; note = uracil
gcaagcagacatttatctt

SEQ ID NO: 52 - DNA, 19 bp, synthetic construct
  source 1..19: mol_type = other DNA
  modified_base 5: mod_base = OTHER; note = uracil
  modified_base 9: mod_base = OTHER; note = uracil
  modified_base 11: mod_base = OTHER; note = uracil
  modified_base 13: mod_base = OTHER; note = uracil
  modified_base 16..17: mod_base = OTHER; note = uracil
aagataaatgtctgcttgc

SEQ ID NO: 53 - DNA, 19 bp, synthetic construct
  source 1..19: mol_type = other DNA
  modified_base 6..8: mod_base = OTHER; note = uracil
  modified_base 14..15: mod_base = OTHER; note = uracil
gggcatttcaccattcaaa

SEQ ID NO: 54 - DNA, 19 bp, synthetic construct
  source 1..19: mol_type = other DNA
  modified_base 1..3: mod_base = OTHER; note = uracil
  modified_base 7: mod_base = OTHER; note = uracil
  modified_base 10: mod_base = OTHER; note = uracil
  modified_base 15: mod_base = OTHER; note = uracil
tttgaatggtgaaatgccc

SEQ ID NO: 55 - DNA, 19 bp, synthetic construct
  source 1..19: mol_type = other DNA
  modified_base 4..6: mod_base = OTHER; note = uracil
  modified_base 12..13: mod_base = OTHER; note = uracil
gcatttcaccattcaaaca

SEQ ID NO: 56 - DNA, 19 bp, synthetic construct
  source 1..19: mol_type = other DNA
  modified_base 1: mod_base = OTHER; note = uracil
  modified_base 3..5: mod_base = OTHER; note = uracil
  modified_base 9: mod_base = OTHER; note = uracil
  modified_base 12: mod_base = OTHER; note = uracil
  modified_base 17: mod_base = OTHER; note = uracil
tgtttgaatggtgaaatgc

SEQ ID NO: 57 - DNA, 19 bp, synthetic construct
  source 1..19: mol_type = other DNA
  modified_base 4..5: mod_base = OTHER; note = uracil
  modified_base 9: mod_base = OTHER; note = uracil
  modified_base 12: mod_base = OTHER; note = uracil
  modified_base 17: mod_base = OTHER; note = uracil
  modified_base 19: mod_base = OTHER; note = uracil
gcattcaatcctcaggtct

SEQ ID NO: 58 - DNA, 19 bp, synthetic construct
  source 1..19: mol_type = other DNA
  modified_base 6: mod_base = OTHER; note = uracil
  modified_base 12..13: mod_base = OTHER; note = uracil
  modified_base 17: mod_base = OTHER; note = uracil
agacctgaggattgaatgc

SEQ ID NO: 59 - DNA, 3637 bp, Homo sapiens
  source 1..3637: mol_type = other DNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa